(19) United States
(12) Patent Application Publication
(10) Pub. No.: US 2019/0169519 A1
(43) Pub. Date: Jun. 6, 2019

(54) MULTIFUNCTIONAL LUBRICATING OIL BASE STOCKS AND PROCESSES FOR PREPARING SAME

(71) Applicant: ExxonMobil Research and Engineering Company, Annandale, NJ (US)

(72) Inventors: Abhimanyu O. PATIL, Westfield, NJ (US); Satish BODIGE, Wayne, NJ (US); Kyle LEWIS, Houston, TX (US)

(21) Appl. No.: 16/211,480

(22) Filed: Dec. 6, 2018

Related U.S. Application Data
(63) Continuation-in-part of application No. 15/198,195, filed on Jun. 30, 2016, now abandoned.
(60) Provisional application No. 62/189,485, filed on Jul. 7, 2015.

(51) Int. Cl. C10M 105/72; C07D 279/20

(52) U.S. Cl. CPC C10M 105/72; C07D 279/20; C10N 2230/08; C10N 2230/10; C10M 2219/108

(57) ABSTRACT

A composition that includes one or more compounds represented by the formula

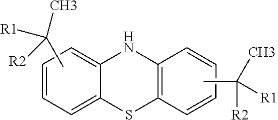

wherein R1 and R2 are same or different and are the residue of an alkyl group having more than 10 carbon atoms wherein the composition has a viscosity (Kv₁₀₀) from 2 to 300 at 100° C. as determined by ASTM D-445, and a viscosity index (VI) from −100 to 300 as determined by ASTM D-2270. The disclosure also relates to a process for producing the composition, a lubricating oil base stock and lubricating oil containing the composition, and a method for improving oxidative stability of a lubricating oil by using as the lubricating oil a formulated oil containing the composition.

### CROSS-REFERENCE TO RELATED APPLICATIONS

This application is a Continuation-in-Part Application and claims priority to pending U.S. application Ser. No. 15/198,195 filed on Jun. 30, 2016, the entirety of which is incorporated herein by reference, which claims priority to U.S. Provisional Application Ser. No. 62/189,485 filed Jul. 7, 2015, which is herein incorporated by reference in its entirety.

### FIELD

This disclosure relates to compositions that include one or more alkylated polycyclic, heteroatom-containing, hydrocarbon compounds (e.g., alkylated phenothiazine and alkylated phenothiazine derivatives), a process for producing the compositions, a lubricating oil base stock and lubricating oil containing the composition, and a method for improving oxidative stability of a lubricating oil by using as the lubricating oil a formulated oil containing the composition.

### BACKGROUND

Lubricants in commercial use today are prepared from a variety of natural and synthetic base stocks admixed with various additive packages and solvents depending upon their intended application. The base stocks typically include mineral oils, polyalphaolefins (PAO), gas-to-liquid base oils (GTL), silicone oils, phosphate esters, diesters, polyol esters, and the like.

A major trend for passenger car engine oils (PCEOs) is an overall improvement in quality as higher quality base stocks become more readily available. Typically the highest quality PCEO products are formulated with base stocks such as PAOs or GTL stocks admixed with various additive packages.

Future automotive and industrial trend suggest that there will be a need for advanced additive technology and synthetic base stocks with substantial better thermal and oxidative stability. This is primarily because of smaller sump sizes that will have more thermal and oxidative stresses on the lubricants. Performance requirements have become more stringent in the past 10 to 20 years and the demand for longer drain intervals has grown steadily. Also, the use of Group II, III and IV base oils is coming more widespread. Such base oils have very little sulfur content since natural sulfur-containing antioxidants are either absent or removed during the severe refining process.

It is known that lubricant oils used in internal combustion engines and transmission of automobile engines or trucks are subjected to demanding environments during use. These environments result in the lubricant suffering oxidation catalyzed by the presence of impurities in the oil, such as iron (wear) compounds and elevated temperatures. The oxidation manifests itself by increase in acid or viscosity and deposit formation or any combination of these symptoms. These are controlled to some extent by the use of antioxidants which can extend the useful life of the lubricating oil, particularly by reducing or preventing unacceptable viscosity increases. Besides oxidation inhibition, other parameters such as rust and wear control are also important.

Therefore, there is need for better additive and base stock technology for lubricant compositions that will meet ever more stringent requirements of lubricant users. In particular, there is a need for advanced additive technology and synthetic base stocks with improved thermal and oxidative stability.

The present disclosure also provides many additional advantages, which shall become apparent as described below.

### SUMMARY

This disclosure relates in part to a composition comprising one or more compounds represented by the formula

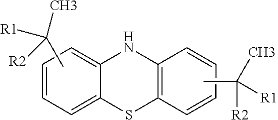

wherein R1 and R2 are same or different and are the residue of an alkyl group having more than 10 carbon atoms. The composition has a viscosity (Kv₁₀₀) from 2 to 300 at 100° C. as determined by ASTM D-445, and a viscosity index (VI) from −100 to 300 as determined by ASTM D-2270.

This disclosure also relates in part to a composition comprising one or more alkylated polycyclic, heteroatom-containing, hydrocarbon compounds. The one or more polycyclic, heteroatom-containing, hydrocarbon compounds are produced by a process that comprises reacting a polyalphaolefin oligomer with at least one polycyclic, heteroatom-containing, hydrocarbon compound, in the presence of a catalyst, under reaction conditions sufficient to produce the one or more alkylated polycyclic, heteroatom-containing, hydrocarbon compounds with a substantially quaternary carbon structure.

This disclosure further relates in part to a process for producing a composition comprising one or more alkylated polycyclic, heteroatom-containing, hydrocarbon compounds. The process comprises reacting a polyalphaolefin oligomer) with at least one polycyclic, heteroatom-containing, hydrocarbon compound, in the presence of a catalyst, under reaction conditions sufficient to produce the composition.

This disclosure yet further relates in part to a lubricating oil base stock comprising one or more compounds represented by the formula

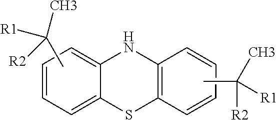

wherein R1 and R2 are same or different and are the residue of an alkyl group having more than 10 carbon atoms. The composition has a viscosity (Kv₁₀₀) from 2 to 300 at 100° C. as determined by ASTM D-445, and a viscosity index (VI) from −100 to 300 as determined by ASTM D-2270.

This disclosure also relates in part to a lubricating oil comprising a lubricating oil base stock as a major component, and a polycyclic, heteroatom-containing, hydrocarbon compound cobase stock as a minor component. The polycyclic, heteroatom-containing, hydrocarbon compound cobase stock comprises one or more compounds represented by the formula

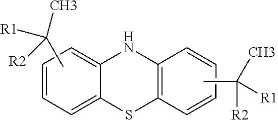

wherein R1 and R2 are same or different and are the residue of an alkyl group having more than 10 carbon atoms. The composition has a viscosity (Kv₁₀₀) from 2 to 300 at 100° C. as determined by ASTM D-445, and a viscosity index (VI) from −100 to 300 as determined by ASTM D-2270.

This disclosure further relates in part to a method for improving oxidative stability of a lubricating oil by using as the lubricating oil a formulated oil comprising a lubricating oil base stock as a major component, and polycyclic, heteroatom-containing, hydrocarbon compound cobase stock as a minor component. The polycyclic, heteroatom-containing, hydrocarbon compound cobase stock comprises one or more compounds represented by the formula

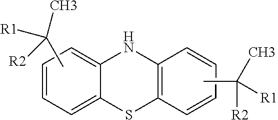

wherein R1 and R2 are same or different and are the residue of an alkyl group having more than 10 carbon atoms. The composition has a viscosity (Kv₁₀₀) from 2 to 300 at 100° C. as determined by ASTM D-445, and a viscosity index (VI) from −100 to 300 as determined by ASTM D-2270 and an oxidation onset temperature (OOT)/oxidation peak temperature (OPT) ranging from about 175° C./325° C. to about 225° C./375° C.

It has been surprisingly found that improved oxidative stability can be attained in an engine lubricated with a lubricating oil by using as the lubricating oil a formulated oil in accordance with this disclosure. In particular, a lubricating oil base stock comprising one or more alkylated polycyclic, heteroatom-containing, hydrocarbon compounds (e.g., alkylated phenothiazine and alkylated phenothiazine derivatives) exhibits superior oxidative stability, which helps to prolong the useful life of lubricants and significantly improve the durability and resistance of lubricants when exposed to high temperatures. The alkylated polycyclic, heteroatom-containing, hydrocarbon compounds (e.g., alkylated phenothiazine and alkylated phenothiazine derivatives) are highly reactive towards radicals and are therefore excellent inhibitors against autoxidation and polymerization. The lubricating oils of this disclosure are particularly advantageous as passenger vehicle engine oil (PVEO) products.

Further objects, features and advantages of the present disclosure will be understood by reference to the following drawings and detailed description.

### BRIEF DESCRIPTION OF THE DRAWINGS

FIG. 1 shows gas chromatographic (GC) analysis of phenothiazine alkylated with mPAO-dimer (C₁₆=) using F24 catalyst in accordance with Example 1.

FIG. 2 shows gel permeation chromatography (GPC) analysis of phenothiazine alkylated with mPAO-dimer (C₁₆=) using F24 catalyst using both differential refractive index (DRI) and ultra violet (UV) detectors in accordance with Example 1.

FIG. 3 shows mass spectrum (MS) of phenothiazine alkylated with mPAO-dimer (C₁₆=) using F24 catalyst using atmospheric pressure photoionization (APPI) in accordance with Example 1.

FIG. 4 shows the ¹H-NMR spectrum of phenothiazine alkylated with mPAO-dimer (C₁₆=) using F24 catalyst in accordance with Example 1.

FIG. 5 shows the ¹³C-NMR and distortionless enhancement by polarization transfer (DEPT) spectrum of phenothiazine alkylated with mPAO-dimer (C₁₆=) using F24 catalyst in accordance with Example 1.

FIG. 6 shows ¹H-NMR spectrum of phenothiazine alkylated with mPAO-dimer (C₁₆=) using MCM49 catalyst in accordance with Example 2. The product was analyzed by GC, ¹H-NMR, ¹³C and distortionless enhancement by polarization transfer (DEPT).

FIG. 7 shows DEPT spectrum of phenothiazine alkylated with mPAO-dimer (C₁₆=) using MCM49 catalyst in accordance with Example 2.

FIG. 8 shows the lube properties and oxidative stability data of the products of Examples 1 and 2 that were evaluated in accordance with Example 3.

FIG. 9 shows ¹H-NMR spectrum of alkylated phenothiazine prepared using MCM49 catalyst in accordance with Example 4.

FIG. 10 shows GC analysis of phenothiazine alkylated mPAO-dimer (C₁₆=) using MCM-49 catalyst product in accordance with Example 4.

FIG. 11 shows GC analysis of fraction 1 of phenothiazine alkylated mPAO-dimer (C₁₆=) using MCM-49 catalyst in accordance with Example 4.

FIG. 12 shows GC analysis of fraction 2 of phenothiazine alkylated with mPAO-dimer (C₁₆=) using MCM-49 catalyst in accordance with Example 4.

FIG. 13 shows the lube properties and data of the products of Examples 6 and 7 that were evaluated in accordance with Example 7.

FIG. 14 shows oxidative stability of two blends measured by rotating pressure vessel oxidation test (RPVOT) in accordance with Example 7. The results are compared against the RPVOT performances of neat SpectraSyn™ 4 PAO and neat C16 mPAO APTZ.

FIG. 15 shows GC analysis of C16 mPAO APTZ (p-TsOH catalyst) in accordance with Example 7.

FIG. 16 shows GC analysis of C12 LAO APTZ (triflic acid catalyst) in accordance with Example 7.

### DETAILED DESCRIPTION

### Definitions

“Substantially” should be understood to mean that the component following the word “substantially” includes only other minor impurity (e.g, at trace amounts) of other components than what is specified after “substantially.”

“About” or “approximately.” All numerical values within the detailed description and the claims herein are modified by “about” or “approximately” the indicated value, and take into account experimental error and variations that would be expected by a person having ordinary skill in the art.

“Major amount” as it relates to components included within the lubricating oils of the specification and the claims means greater than or equal to 50 wt. %, or greater than or equal to 60 wt. %, or greater than or equal to 70 wt. %, or greater than or equal to 80 wt. %, or greater than or equal to 90 wt. % based on the total weight of the lubricating oil.

“Minor amount” as it relates to components included within the lubricating oils of the specification and the claims means less than 50 wt. %, or less than or equal to 40 wt. %, or less than or equal to 30 wt. %, or greater than or equal to 20 wt. %, or less than or equal to 10 wt. %, or less than or equal to 5 wt. %, or less than or equal to 2 wt. %, or less than or equal to 1 wt. %, based on the total weight of the lubricating oil.

“Essentially free” as it relates to components included within the lubricating oils of the specification and the claims means that the particular component is at 0 weight % within the lubricating oil, or alternatively is at impurity type levels within the lubricating oil (less than 100 ppm, or less than 20 ppm, or less than 10 ppm, or less than 1 ppm).

“Other lubricating oil additives” as used in the specification and the claims means other lubricating oil additives that are not specifically recited in the particular section of the specification or the claims. For example, other lubricating oil additives may include, but are not limited to, antioxidants, detergents, dispersants, antiwear additives, corrosion inhibitors, viscosity modifiers, metal passivators, pour point depressants, seal compatibility agents, antifoam agents, extreme pressure agents, friction modifiers and combinations thereof.

“Hydrocarbon” refers to a compound consisting of carbon atoms and hydrogen atoms.

“Alkane” refers to a hydrocarbon that is completely saturated. An alkane can be linear, branched, cyclic, or substituted cyclic.

“Olefin” refers to a non-aromatic hydrocarbon comprising one or more carbon-carbon double bond in the molecular structure thereof.

“Mono-olefin” refers to an olefin comprising a single carbon-carbon double bond.

“Cn” group or compound refers to a group or a compound comprising carbon atoms at total number thereof of n. Thus, “Cm-Cn” group or compound refers to a group or compound comprising carbon atoms at a total number thereof in the range from m to n. Thus, a C1-C50 alkyl group refers to an alkyl group comprising carbon atoms at a total number thereof in the range from 1 to 50.

“Carbon backbone” refers to the longest straight carbon chain in the molecule of the compound or the group in question. “Branch” refer to any substituted or unsubstituted hydrocarbyl group connected to the carbon backbone. A carbon atom on the carbon backbone connected to a branch is called a “branched carbon.”

“Epsilon-carbon” in a branched alkane refers to a carbon atom in its carbon backbone that is (i) connected to two hydrogen atoms and two carbon atoms and (ii) connected to a branched carbon via at least four (4) methylene (CH₂) groups. Quantity of epsilon carbon atoms in terms of mole percentage thereof in a alkane material based on the total moles of carbon atoms can be determined by using, e.g., ¹³C NMR.

“SAE” refers to SAE International, formerly known as Society of Automotive Engineers, which is a professional organization that sets standards for internal combustion engine lubricating oils.

“SAE J300” refers to the viscosity grade classification system of engine lubricating oils established by SAE, which defines the limits of the classifications in rheological terms only.

“Base stock” or “base oil” interchangeably refers to an oil that can be used as a component of lubricating oils, heat transfer oils, hydraulic oils, grease products, and the like.

“Lubricating oil” or “lubricant” interchangeably refers to a substance that can be introduced between two or more surfaces to reduce the level of friction between two adjacent surfaces moving relative to each other. A lubricant base stock is a material, typically a fluid at various levels of viscosity at the operating temperature of the lubricant, used to formulate a lubricant by admixing with other components. Non-limiting examples of base stocks suitable in lubricants include API Group I, Group II, Group III, Group IV, and Group V base stocks. PAOs, particularly hydrogenated PAOs, have recently found wide use in lubricants as a Group IV base stock, and are particularly preferred. If one base stock is designated as a primary base stock in the lubricant, additional base stocks may be called a co-base stock.

All kinematic viscosity values in this disclosure are as determined pursuant to ASTM D445. Kinematic viscosity at 100° C. is reported herein as KV100, and kinematic viscosity at 40° C. is reported herein as KV40. Unit of all KV100 and KV40 values herein is cSt unless otherwise specified.

All viscosity index (“VI”) values in this disclosure are as determined pursuant to ASTM D2270.

All Noack volatility (“NV”) values in this disclosure are as determined pursuant to ASTM D5800 unless specified otherwise. Unit of all NV values is wt %, unless otherwise specified.

All pour point values in this disclosure are as determined pursuant to ASTM D5950 or D97.

All CCS viscosity (“CCSV”) values in this disclosure are as determined pursuant to ASTM 5293. Unit of all CCSV values herein is millipascal second (mPa·s), which is equivalent to centipoise), unless specified otherwise. All CCSV values are measured at a temperature of interest to the lubricating oil formulation or oil composition in question. Thus, for the purpose of designing and fabricating engine oil formulations, the temperature of interest is the temperature at which the SAE J300 imposes a minimal CCSV.

All percentages in describing chemical compositions herein are by weight unless specified otherwise. “Wt. %” means percent by weight.

Compositions and Methods of this Disclosure

Lubricants, like all organic materials, are prone to thermo-oxidative degradation. Fortunately, this degradation can be minimized or retarded. Primary and secondary antioxidants are used successfully to improve the oxidative stability of oils exposed to high temperatures and to extend their service life.

Types of antioxidants typically used are radical scavengers (primary antioxidants) such as aromatic amines and hindered phenols, and peroxide decomposers (secondary antioxidants) such as organophosphorus compounds and organosulfur compounds.

Primary antioxidants slow down the thermo-oxidative degradation chain process by trapping radicals.

R·+AH→RH+A·

Secondary antioxidants react with peroxides (ROOR’/ROOH).

RSR+R′OOH→RS(═O)R+R′OH

The efficiency of an antioxidant depends upon the structure. For primary antioxidants, such as aminic antioxidants, the dissociation emery of the N-H bond, E_{(N-H)}, is a key parameter. Amines which contain sulfur (e.g., alkylated phenothiazine) have a lower bond dissociation energy (i.e. higher rate constant) than alkylated diphenylamines (ADPA). A decrease of E_{(N-H)} by a few kJ/mol can increase the rate constant by a factor of 10. The N-H bond dissociation energy of antioxidants is shown below.

| | Alkylated | Alkylated |
|---|---|---|
| Antioxidant structure | phenothiazine | diphenylamine |
| E_{(N—H)} in kJ/mol | 326 | 356 |

As indicated above, the compositions of this disclosure comprise one or more compounds represented by the formula

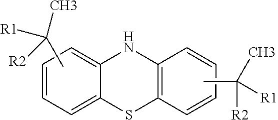

wherein R1 and R2 are same or different and are the residue of an alkyl group having more than 10 carbon atoms. The composition has a viscosity (Kv₁₀₀) from 2 to 300 at 100° C. as determined by ASTM D-445, and a viscosity index (VI) from −100 to 300 as determined by ASTM D-2270.

Alternatively R1 and R2 are same or different and are the residue of an alkyl group having more than 10 carbon atoms and less than or equal to 140 carbon atoms. Still alternatively R1 and R2 are same or different and are the residue of an alkyl group having more than 10 carbon atoms, or greater than or equal to 12 carbon atoms, or greater than or equal to 14 carbon atoms, or greater than or equal to 16 carbon atoms, or greater than or equal to 18 carbon atoms, or greater than or equal to 20 carbon atoms, or greater than or equal to 22 carbon atoms, or greater than or equal to 24 carbon atoms, or greater than or equal to 26 carbon atoms. Still more alternatively R1 and R2 are same or different and are the residue of an alkyl group having less than or equal to 140 carbon atoms, or less than or equal to 120 carbon atoms, or less than or equal to 100 carbon atoms, or less than or equal to 80 carbon atoms, or less than or equal to 60 carbon atoms, or less than or equal to 50 carbon atoms, or less than or equal to 40 carbon atoms, or less than or equal to 30 carbon atoms.

Illustrative R₁ and R₂ substituents include, for example, C₁₀-C₁₄₀ alkane hydrocarbons, the residue of mPAO dimers (C₁₂-C₂₈), trimers (C₁₈-C₄₂), and higher oligomers, pentamer, hexamer, and the like. Preferably, R₁ and R₂ are independently the residue of a mPAO trimer, more preferably a mPAO dimer (C₁₂, C₁₆, C₂₀, C₂₄ or C₂₈) that has a substantially vinylidene double bond.

Illustrative compositions of this disclosure include, for example, polycyclic, heteroatom-containing, hydrocarbon compound-containing mPAO dimers, trimers, tetramers, pentamers, hexamers, and higher oligomers, . Preferred compositions result from selective coupling of mPAO dimer (e.g., mPAO 1-decene) with a polycyclic, heteroatom-containing, hydrocarbon compound (e.g., alkylated phenothiazine and alkylated phenothiazine derivatives).

In an embodiment, the compositions of this disclosure comprise one or more compounds represented by the formula

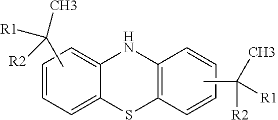

wherein R1 and R2 are same or different and are the residue of an alkyl group having more than 10 carbon atoms wherein the composition has a viscosity (Kv₁₀₀) from 2 to 300 at 100° C. as determined by ASTM D-445, and a viscosity index (VI) from −100 to 300 as determined by ASTM D-2270.

In particular, compositions of this disclosure include, for example, the reaction product of phenothiazine with mPAO dimer, the reaction of a phenothiazine derivative with mPAO dimer, and the like.

The composition of this disclosure can be prepared by a process that involves reacting a polyalphaolefin oligomer with at least one polycyclic, heteroatom-containing, hydrocarbon compound. The reaction is carried out in the presence of a catalyst. The reaction is also carried out under reaction conditions sufficient to produce the composition.

Illustrative polyalphaolefin oligomers useful in the process of this disclosure include, for example, mPAO dimers, trimers, tetramers, higher oligomers, and the like.

In an embodiment, the mPAO dimer can be any dimer prepared from metallocene or other single-site catalyst with terminal double bond. The dimer can be from 1-decene, 1-octene, 1-dodecene, 1-hexene, 1-tetradecene, 1-octadecene or combination of alpha-olefins. The general alkylation reaction scheme is shown below.

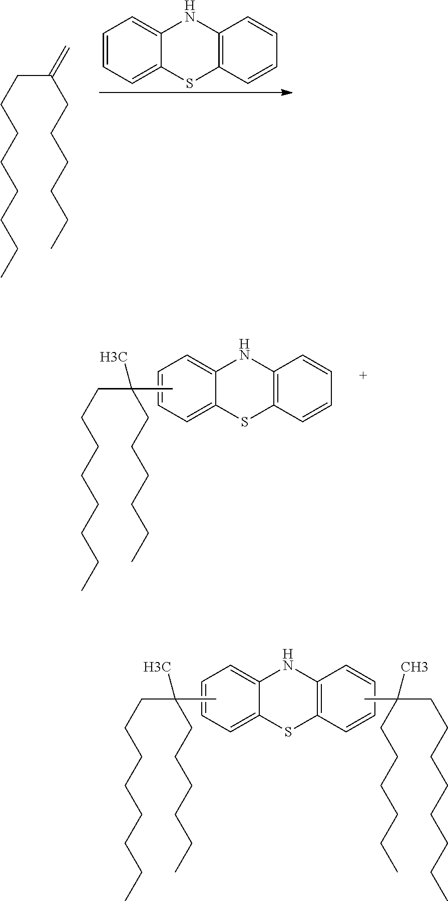

The alkyl group derived from umPAO dimer (C12=, C16=, C20=, C24=, C28=) or trimer (C18=, C24=, C30=, C36=, C42=) that has a substantially vinylidene double bond. The alkylated phenothiazine that results in product with a substantially quaternary carbon structure. The product can be used as high temperature basestock. umPAO can be higher molecular weight PAO such as unhydrogenated 65 cSt PAO. umPAO could have a range of olefin geometries, for example, it could be a mixture of vinylidene or trisubstituted olefin, with minor components of internal olefins.

The olefin feed useful in the process of this disclosure can include a light olefinic byproduct fraction including dimers and light fractions from the metallocene-catalyzed PAO oligomerization process. These intermediate light fractions may be generally characterized as C₄₂ or lower olefinic distillate fractions that contain a mixture of highly reactive oligomers derived from the original alpha-olefin starting material.

The metallocene-derived intermediate useful as a feed material is produced by the oligomerization of an alpha-olefin feed using a metallocene oligomerization catalyst. The alpha olefin feeds used in this initial oligomerization step are typically alpha-olefin monomers of 4 to 24 carbon atoms, usually 6 to 20 and preferably 8 to 14 carbon atoms. Illustrative alpha olefin feeds include, for example, 1-butene, 1-hexene, 1-octene, 1-decene, 1-dodecene, 1-tetradecene, and the like. The olefins with even carbon numbers are preferred as are the linear alpha-olefins, although it is possible to use branched-chain olefins containing an alkyl substituent at least two carbons away from the terminal double bond.

The initial oligomerization step using a metallocene catalyst can be carried out under the conditions appropriate to the selected alpha-olefin feed and metallocene catalyst. A preferred metallocene-catalyzed alpha-olefin oligomerization process is described in WO 2007/011973, which is incorporated herein by reference in its entirety and to which reference is made for details of feeds, metallocene catalysts, process conditions and characterizations of products.

The dimers useful as feeds in the process of this disclosure possess at least one carbon-carbon unsaturated double bond. The unsaturation is normally more or less centrally located at the junction of the two monomer units making up the dimer as a result of the non-isomerizing polymerization mechanism characteristic of metallocene processes. If the initial metallocene polymerization step uses a single 1-olefin feed to make an alpha-olefin homopolymer, the unsaturation will be centrally located but if two 1-olefin comonomers have been used to form a metallocene copolymer, the location of the double bond may be shifted off center in accordance with the chain lengths of the two comonomers used. In any event, this double bond is 1,2-substituted internal, vinylic or vinylidenic in character. The terminal vinylidene group is represented by the formula RₐR_{b}C═CH₂, referred to as vinyl when the formula is RₐHC═CH₂. The amount of unsaturation can be quantitatively measured by bromine number measurement according to ASTM D1159 or equivalent method, or according to proton or carbon-13 NMR. Proton NMR spectroscopic analysis can also differentiate and quantify the types of olefinic unsaturation.

For alkylation reaction, illustrative olefins that can be used include, for example, unhydrogenated poly-a-olefins, unhydrogenated ethylene a-olefin copolymers, unhydrogenated polyisobutylene, olefins with terminal double bond containing macromers, and the like.

Illustrative polycyclic, heteroatom-containing, hydrocarbon compounds useful in the process of this disclosure include, for example, phenothiazine, derivatives of phenothiazine, 2-methylthiophenothiazine, 10-methylphenothiazine, monotetradecylphenothiazine, ditetradecylphenothiazine, monodecylphenothiazine, didecylphenothiazine, monononylphenothiazine, dinonylphenothiazine, monoctylphenothiazine, dioctylphenothiazine, monobutylphenothiazine, dibutylphenothiazine, monostyrylphenothiazine, distyrylphenothiazine, butyloctylphenothiazine, styryloctylphenothiazine, and mixtures thereof, and the like. This disclosure encompasses the whole group of polycyclic, heteroatom-containing, hydrocarbon compounds.

In an embodiment, the polycyclic, heteroatom-containing, hydrocarbon compounds useful in the process of this disclosure comprise one or more compounds represented by the formula

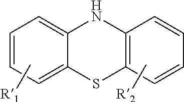

wherein R′¹ and R′₂ are the same or different and are hydrogen or the residue of an alkyl group having from about 1 to about 40 carbon atoms.

Illustrative catalysts that can be used in the process of this disclosure include, for example, Bronsted acids, Lewis acids, aluminum silicates, ion exchange resins, zeolites, naturally occurring sheet silicates, modified sheet silicates and acidic ionic liquids, and the like.

In particular, the acid catalyst is preferably acid clay of the bentonire or montmorillonite type. The reaction can be catalyzed homogeneously or heterogeneously. Illustrative examples of suitable Bronsted acids are acids of inorganic or organics salts, typically hydrochloric acid, sulfuric acid, phosphoric acid, methanesulfonic acid, p-toluenesulfonic acid, or carboxylic acids such as acetic acid. p-Toluenesulfonic acid is particularly suitable. Illustrative examples of suitable Lewis acids are tin tetrachloride, aluminum chloride, zinc chloride or boron trifluoride etherate. Tin tetrachloride and aluminum chloride are particularly suitable. Illustrative examples of suitable aluminum silicates are those that are widely used in the petrochemical industry and are also known as amorphous aluminum silicates. These compounds contain about 10-30% of silicon monoxide and 70-90% of aluminum oxide. A particularly preferred aluminum silicate is HA-HPV® available from Ketjen (Akzo).

Illustrative examples of suitable ion exchange resins are typically styrene-divinylbenzene resins which additionally carry sulfonic acid groups, for example, Amberlite 200® and Amberlyst available from Rohm and Haas, or Dowex 50® available from Dow Chemicals; perfluorinated ion exchange resins such as Nafion HO sold by DuPont; or other superacidic ion exchange resins such as those described by T. Yamaguchi, Applied Catalysis, 61, 1-25 (1990) or M. Hino et al., J. Chem. Soc. Chem. Commun. 1980, 851-852. Suitable zeolites are typically those widely used in petrochemistry as cracking catalysts and known as crystalline silicon-aluminum oxides of different crystal structure. Particularly preferred zeolites are the Faujasites available from Union Carbide, for example Zeolith X©, Zeolith Y® and ultrastable Zeolith Y®; Zeolith Beta® and Zeolith ZSM-12® available from Mobil Oil Co.; and Zeolith Moralenit available from Norton.

Suitable naturally occurring sheet silicates are also termed “acid clays” and typically include bentonites or montmorillonites, which are degraded, ground, treated with mineral acids and calcined industrially. Particularly suitable natural sheet silicates are Fulcat types available from Laporte Adsorbents Co., for example Fulcat 22A®, Fulcat 22B®, Fulcat 20® or Fulcat 40®; or the Fulmont® types available from Laporte Adsorbents Co., for example Fulmont XMP-3® or Fulmont XMP-4®. A particularly preferred catalyst for the process of this disclosure is Fulcat 22B®, an acid activated montmorillonite having 4% of free moisture and an acid titer of 20 mg KOH/g. However, the other Fulcat types and Fulmont® types also belong to this preferred class, because there are only minor differences between the individual types, as for example in the number of acid centers.

Modified sheet silicates are also termed “pillared clays” and are derived from the above described naturally occurring sheet silicates by additionally containing between the silicate layers oxides of (e.g., zirconium, iron, zinc, nickel, chromium, cobalt or magnesium, or elements of the rare earths). This type of catalyst is described in the literature, inter alia by J. Clark et. al., J. Chem. Soc. Chem. Commun. 1989, 1353-1354 and is widely used, but is available from only a very few firms. Particularly preferred modified sheet silicams typically include Envirocat EPZ-10©, Envirocat EPZG® or Envirocat EPIC available from Contract Chemicals.

Reaction conditions for the reaction of the polyalphaolefin oligomer with the polycyclic, heteroatom-containing, hydrocarbon compound (e.g., phenothiazine, a derivative of phenothiazine, or mixtures thereof), such as temperature, pressure and contact time, may also vary greatly and any suitable combination of such conditions may be employed herein. The reaction temperature may range between about 25° C. to about 250° C., and preferably between about 30° C. to about 200° C., and more preferably between about 60° C. to about 150° C. Normally the reaction is carried out under ambient pressure and the contact time may vary from a matter of seconds or minutes to a few hours or greater. The reactants can be added to the reaction mixture or combined in any order. The stir time employed can range from about 0.5 to about 48 hours, preferably from about 1 to 36 hours, and more preferably from about 2 to 24 hours.

In an embodiment, the process of this disclosure involves selective coupling of mPAO (metallocene polyalphaolefin) 1-decene dimer with a polycyclic, heteroatom-containing, hydrocarbon compound (e.g., phenothiazine, a derivative of phenothiazine, or mixtures thereof) that can render unique lube properties (i.e., oxidative stability). For example, a mPAO dimer (C₁₆=, C₂₀=) can be reacted with phenothiazine to give a synthetic base stock.

Examples of techniques that can be employed to characterize the compositions formed by the process described above include, but are not limited to, analytical gas chromatography, nuclear magnetic resonance, thermogravimetric analysis (TGA), inductively coupled plasma mass spectrometry, differential scanning calorimetry (DSC), volatility and viscosity measurements.

This disclosure provides lubricating oils useful as engine oils and in other applications characterized by excellent oxidative stability. The lubricating oils are based on high quality base stocks including a major portion of a hydrocarbon base fluid such as a PAO or GTL with a secondary cobase stock component which is a polycyclic, heteroatom-containing, hydrocarbon compound (e.g., phenothiazine, a derivative of phenothiazine, or mixtures thereof) as described herein. The lubricating oil base stock can be any oil boiling in the lube oil boiling range, typically between about 100 to 450° C. In the present specification and claims, the terms base oil(s) and base stock(s) are used interchangeably.

The viscosity-temperature relationship of a lubricating oil is one of the critical criteria which must be considered when selecting a lubricant for a particular application. Viscosity Index (VI) is an empirical, unitless number which indicates the rate of change in the viscosity of an oil within a given temperature range. Fluids exhibiting a relatively large change in viscosity with temperature are said to have a low viscosity index. A low VI oil, for example, will thin out at elevated temperatures faster than a high VI oil. Usually, the high VI oil is more desirable because it has higher viscosity at higher temperature, which translates into better or thicker lubrication film and better protection of the contacting machine elements.

In another aspect, as the oil operating temperature decreases, the viscosity of a high VI oil will not increase as much as the viscosity of a low VI oil. This is advantageous because the excessive high viscosity of the low VI oil will decrease the efficiency of the operating machine. Thus high VI (HVI) oil has performance advantages in both high and low temperature operation. VI is determined according to ASTM method D 2270-93 [1998]. VI is related to kinematic viscosities measured at 40° C. and 100° C. using ASTM Method D 445-01.

### Other Lubricating Oil Base Stocks

A wide range of lubricating oils is known in the art. Lubricating oils that are useful in the present disclosure are both natural oils and synthetic oils. Natural and synthetic oils (or mixtures thereof) can be used unrefined, refined, or rerefined (the latter is also known as reclaimed or reprocessed oil). Unrefined oils are those obtained directly from a natural or synthetic source and used without added purification. These include shale oil obtained directly from retorting operations, petroleum oil obtained directly from primary distillation, and ester oil obtained directly from an esterification process. Refined oils are similar to the oils discussed for unrefined oils except refined oils are subjected to one or more purification steps to improve the at least one lubricating oil property. One skilled in the art is familiar with many purification processes. These processes include solvent extraction, secondary distillation, acid extraction, base extraction, filtration, and percolation. Rerefined oils are obtained by processes analogous to refined oils but using an oil that has been previously used as a feed stock.

Groups I, II, III, IV and V are broad categories of base oil stocks developed and defined by the American Petroleum Institute (API Publication 1509; www.API.org) to create guidelines for lubricant base oils. Group I base stocks generally have a viscosity index of between about 80 to 120 and contain greater than about 0.03% sulfur and less than about 90% saturates. Group II base stocks generally have a viscosity index of between about 80 to 120, and contain less than or equal to about 0.03% sulfur and greater than or equal to about 90% saturates. Group III stock generally has a viscosity index greater than about 120 and contains less than or equal to about 0.03% sulfur and greater than about 90% saturates. Group IV includes polyalphaolefins (PAO). Group V base stocks include base stocks not included in Groups I-IV. The table below summarizes properties of each of these five groups.

| | Base Oil Properties | | |
|---|---|---|---|
| | Saturates | Sulfur | Viscosity Index |
| Group I | <90 and/or | >0.03% and | ≥80 and <120 |
| Group II | ≥90 and | ≤0.03% and | ≥80 and <120 |
| Group III | ≥90 and | ≤0.03% and | ≥120 |

| | |
|---|---|
| Group IV | Includes polyalphaolefins (PAO) products |
| Group V | All other base oil stocks not |
| | included in Groups I, II, III or IV |

Natural oils include animal oils, vegetable oils (castor oil and lard oil, for example), and mineral oils. Animal and vegetable oils possessing favorable thermal oxidative stability can be used. Of the natural oils, mineral oils are preferred. Mineral oils vary widely as to their crude source, for example, as to whether they are paraffinic, naphthenic, or mixed paraffinic-naphthenic. Oils derived from coal or shale are also useful in the present disclosure. Natural oils vary also as to the method used for their production and purification, for example, their distillation range and whether they are straight run or cracked, hydrorefined, or solvent extracted.

Group II and/or Group III hydroprocessed or hydrocracked base stocks, as well as synthetic oils such as polyalphaolefins, alkyl aromatics and synthetic esters, i.e. Group IV and Group V oils are also well known base stock oils.

Synthetic oils include hydrocarbon oil such as polymerized and interpolymerized olefins (polybutylenes, polypropylenes, propylene isobutylene copolymers, ethylene-olefin copolymers, and ethylene-alphaolefin copolymers, for example). Polyalphaolefin (PAO) oil base stocks, the Group IV API base stocks, are a commonly used synthetic hydrocarbon oil. By way of example, PAOs derived from C₈, C₁₀, C₁₂, C₁₄ olefins or mixtures thereof may be utilized. See U.S. Pat. Nos. 4,956,122; 4,827,064; and 4,827,073, which are incorporated herein by reference in their entirety. Group IV oils, that is, the PAO base stocks have viscosity indices preferably greater than 130, more preferably greater than 135, still more preferably greater than 140.

Esters in a minor amount may be useful in the lubricating oils of this disclosure. Additive solvency and seal compatibility characteristics may be secured by the use of esters such as the esters of dibasic acids with monoalkanols and the polyol esters of monocarboxylic acids. Esters of the former type include, for example, the esters of dicarboxylic acids such as phthalic acid, succinic acid, sebacic acid, fumaric acid, adipic acid, linoleic acid dimer, malonic acid, alkyl malonic acid, alkenyl malonic acid, etc., with a variety of alcohols such as butyl alcohol, hexyl alcohol, dodecyl alcohol, 2-ethylhexyl alcohol, etc. Specific examples of these types of esters include dibutyl adipate, di(2-ethylhexyl) sebacate, di-n-hexyl fumarate, dioctyl sebacate, diisooctyl azelate, diisodecyl azelate, dioctyl phthalate, didecyl phthalate, dieicosyl sebacate, etc.

Particularly useful synthetic esters are those which are obtained by reacting one or more polyhydric alcohols, preferably the hindered polyols such as the neopentyl polyols; e.g., neopentyl glycol, trimethylol ethane, 2-methyl-2-propyl-1,3-propanediol, trimethylol propane, pentaerythritol and dipentaerythritol with alkanoic acids containing at least about 4 carbon atoms, preferably C₅ to C₃₀ acids such as saturated straight chain fatty acids including caprylic acid, capric acids, lauric acid, myristic acid, palmitic acid, stearic acid, arachic acid, and behenic acid, or the corresponding branched chain fatty acids or unsaturated fatty acids such as oleic acid, or mixtures of any of these materials.

Esters should be used in an amount such that the improved wear and corrosion resistance provided by the lubricating oils of this disclosure are not adversely affected.

Non-conventional or unconventional base stocks and/or base oils include one or a mixture of base stock(s) and/or base oil(s) derived from: (1) one or more Gas-to-Liquids (GTL) materials, as well as (2) hydrodewaxed, or hydroisomerized/cat (and/or solvent) dewaxed base stock(s) and/or base oils derived from synthetic wax, natural wax or waxy feeds, mineral and/or non-mineral oil waxy feed stocks such as gas oils, slack waxes (derived from the solvent dewaxing of natural oils, mineral oils or synthetic oils; e.g., Fischer-Tropsch feed stocks), natural waxes, and waxy stocks such as gas oils, waxy fuels hydrocracker bottoms, waxy raffinate, hydrocrackate, thermal crackates, foots oil or other mineral, mineral oil, or even non-petroleum oil derived waxy materials such as waxy materials recovered from coal liquefaction or shale oil, linear or branched hydrocarbyl compounds with carbon number of about 20 or greater, preferably about 30 or greater and mixtures of such base stocks and/or base oils.

GTL materials are materials that are derived via one or more synthesis, combination, transformation, rearrangement, and/or degradation/deconstructive processes from gaseous carbon-containing compounds, hydrogen-containing compounds and/or elements as feed stocks such as hydrogen, carbon dioxide, carbon monoxide, water, methane, ethane, ethylene, acetylene, propane, propylene, propyne, butane, butylenes, and butynes. GTL base stocks and/or base oils are GTL materials of lubricating viscosity that are generally derived from hydrocarbons; for example, waxy synthesized hydrocarbons, that are themselves derived from simpler gaseous carbon-containing compounds, hydrogen-containing compounds and/or elements as feed stocks. GTL base stock(s) and/or base oil(s) include oils boiling in the lube oil boiling range (1) separated/fractionated from synthesized GTL materials such as, for example, by distillation and subsequently subjected to a final wax processing step which involves either or both of a catalytic dewaxing process, or a solvent dewaxing process, to produce lube oils of reduced/low pour point; (2) synthesized wax isomerates, comprising, for example, hydrodewaxed or hydroisomerized cat and/or solvent dewaxed synthesized wax or waxy hydrocarbons; (3) hydrodewaxed or hydroisomerized cat and/or solvent dewaxed Fischer-Tropsch (F-T) material (i.e., hydrocarbons, waxy hydrocarbons, waxes and possible analogous oxygenates); preferably hydrodewaxed or hydroisomerized/followed by cat and/or solvent dewaxing dewaxed F-T waxy hydrocarbons, or hydrodewaxed or hydroisomerized/followed by cat (or solvent) dewaxing dewaxed, F-T waxes, or mixtures thereof.

GTL base stock(s) and/or base oil(s) derived from GTL materials, especially, hydrodewaxed or hydroisomerized/followed by cat and/or solvent dewaxed wax or waxy feed, preferably F-T material derived base stock(s) and/or base oil(s), are characterized typically as having kinematic viscosities at 100° C. of from about 2 mm²/s to about 50 mm²/s (ASTM D445). They are further characterized typically as having pour points of −5° C. to about −40° C. or lower (ASTM D97). They are also characterized typically as having viscosity indices of about 80 to about 140 or greater (ASTM D2270).

In addition, the GTL base stock(s) and/or base oil(s) are typically highly paraffinic (>90% saturates), and may contain mixtures of monocycloparaffins and multicycloparaffins in combination with non-cyclic isoparaffins. The ratio of the naphthenic (i.e., cycloparaffin) content in such combinations varies with the catalyst and temperature used. Further, GTL base stock(s) and/or base oil(s) typically have very low sulfur and nitrogen content, generally containing less than about 10 ppm, and more typically less than about 5 ppm of each of these elements. The sulfur and nitrogen content of GTL base stock(s) and/or base oil(s) obtained from F-T material, especially F-T wax, is essentially nil. In addition, the absence of phosphorous and aromatics make this materially especially suitable for the formulation of low SAP products.

The term GTL base stock and/or base oil and/or wax isomerate base stock and/or base oil is to be understood as embracing individual fractions of such materials of wide viscosity range as recovered in the production process, mixtures of two or more of such fractions, as well as mixtures of one or two or more low viscosity fractions with one, two or more higher viscosity fractions to produce a blend wherein the blend exhibits a target kinematic viscosity.

The GTL material, from which the GTL base stock(s) and/or base oil(s) is/are derived is preferably an F-T material (i.e., hydrocarbons, waxy hydrocarbons, wax).

Base oils for use in the formulated lubricating oils useful in the present disclosure are any of the variety of oils corresponding to API Group I, Group II, Group III, Group IV, Group V and Group VI oils and mixtures thereof, preferably API Group II, Group III, Group IV, Group V and Group VI oils and mixtures thereof, more preferably the Group III to Group VI base oils due to their exceptional volatility, stability, viscometric and cleanliness features. Minor quantities of Group I stock, such as the amount used to dilute additives for blending into formulated lube oil products, can be tolerated but should be kept to a minimum, i.e. amounts only associated with their use as diluent/carrier oil for additives used on an “as received” basis. Even in regard to the Group II stocks, it is preferred that the Group II stock be in the higher quality range associated with that stock, i.e. a Group II stock having a viscosity index in the range 100<VI<120.

In addition, the GTL base stock(s) and/or base oil(s) are typically highly paraffinic (>90% saturates), and may contain mixtures of monocycloparaffins and multicycloparaffins in combination with non-cyclic isoparaffins. The ratio of the naphthenic (i.e., cycloparaffin) content in such combinations varies with the catalyst and temperature used. Further, GTL base stock(s) and/or base oil(s) and hydrodewaxed, or hydroisomerized/cat (and/or solvent) dewaxed base stock(s) and/or base oil(s) typically have very low sulfur and nitrogen content, generally containing less than about 10 ppm, and more typically less than about 5 ppm of each of these elements. The sulfur and nitrogen content of GTL base stock(s) and/or base oil(s) obtained from F-T material, especially F-T wax, is essentially nil. In addition, the absence of phosphorous and aromatics make this material especially suitable for the formulation of low sulfur, sulfated ash, and phosphorus (low SAP) products.

The base stock component of the present lubricating oils will typically be from 50 to 99 weight percent of the total composition (all proportions and percentages set out in this specification are by weight unless the contrary is stated) and more usually in the range of 80 to 99 weight percent.

### Alkylated Polycyclic, Heteroatom-Containing, Hydrocarbon Compound Base Stock and Cobase Stock Components

Alkylated polycyclic, heteroatom-containing, hydrocarbon compound base stock and cobase stock components useful in this disclosure include, for example, compositions containing one or more compounds represented by the formula

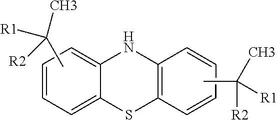

wherein R1 and R2 are same or different and are the residue of an alkyl group having more than 10 carbon atoms wherein the composition has a viscosity (Kv₁₀₀) from 2 to 300 at 100° C. as determined by ASTM D-445, and a viscosity index (VI) from −100 to 300 as determined by ASTM D-2270.

Illustrative alkylated polycyclic, heteroatom-containing, hydrocarbon compound base stock and cobase stock components useful in the present disclosure include, for example, the product of a mPAO dimer (C₁₆═, C₂₀═) reacted with phenothiazine, a derivative of phenothiazine, or mixtures thereof, and the like.

In an embodiment, the alkylated polycyclic, heteroatom-containing, hydrocarbon compound base stock and cobase stock components useful in the present disclosure comprise one or more compounds represented by the formula

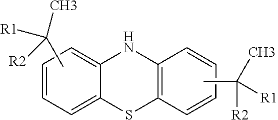

wherein R1 and R2 are same or different and are the residue of an alkyl group having more than 10 carbon atoms wherein the composition has a viscosity (Kv₁₀₀) from 2 to 300 at 100° C. as determined by ASTM D-445, and a viscosity index (VI) from −100 to 300 as determined by ASTM D-2270 and an oxidation onset temperature (OOT)/oxidation peak temperature (OPT) ranging from about 175° C./325° C. to about 225° C./375° C.

Methods for the production of alkylated polycyclic, heteroatom-containing, hydrocarbon compound base stock and cobase stock components suitable for use in the present disclosure are described herein. For example, a polyalphaolefin oligomer can be reacted with an polycyclic, heteroatom-containing, hydrocarbon compound. The reaction is carried out in the presence of a catalyst. The reaction is carried out under reaction conditions sufficient to produce the alkylated polycyclic, heteroatom-containing, hydrocarbon compound base stock and cobase stock as more fully described hereinabove.

The alkylated polycyclic, heteroatom-containing, hydrocarbon compound cobase stock component is preferably present in an amount sufficient for providing oxidative stability in the lubricating oil. The alkylated polycyclic, heteroatom-containing, hydrocarbon compound cobase stock component is present in the lubricating oils of this disclosure in an amount from about 1 to about 50 weight percent, preferably from about 5 to about 30 weight percent, and more preferably from about 10 to about 20 weight percent.

The alkylated polycyclic, heteroatom-containing, hydrocarbon compound base stock component of the present lubricating oils will typically be from 20 to 80 weight percent or from 50 to 99 weight percent of the total composition (all proportions and percentages set out in this specification are by weight unless the contrary is stated) and more usually in the range of 80 to 99 weight percent.

### Other Additives

The formulated lubricating oil useful in the present disclosure may additionally contain one or more of the other commonly used lubricating oil performance additives including but not limited to dispersants, other detergents, corrosion inhibitors, rust inhibitors, metal deactivators, other anti-wear agents and/or extreme pressure additives, anti-seizure agents, wax modifiers, viscosity index improvers, viscosity modifiers, fluid-loss additives, seal compatibility agents, other friction modifiers, lubricity agents, anti-staining agents, chromophoric agents, defoamants, demulsifiers, emulsifiers, densifiers, wetting agents, gelling agents, tackiness agents, colorants, and others. For a review of many commonly used additives, see Klamann in Lubricants and Related Products, Verlag Chemie, Deerfield Beach, FL; ISBN 0-89573-177-0. Reference is also made to “Lubricant Additives Chemistry and Applications” edited by Leslie R. Rudnick, Marcel Dekker, Inc. New York, 2003 ISBN: 0-8247-0857-1.

The types and quantities of performance additives used in combination with the instant disclosure in lubricant compositions are not limited by the examples shown herein as illustrations.

### Viscosity Improvers

Viscosity improvers (also known as Viscosity Index modifiers, and VI improvers) increase the viscosity of the oil composition at elevated temperatures which increases film thickness, while having limited effect on viscosity at low temperatures.

Suitable viscosity improvers include high molecular weight hydrocarbons, polyesters and viscosity index improver dispersants that function as both a viscosity index improver and a dispersant. Typical molecular weights of these polymers are between about 10,000 to 1,000,000, more typically about 20,000 to 500,000, and even more typically between about 50,000 and 200,000.

Examples of suitable viscosity improvers are polymers and copolymers of methacrylate, butadiene, olefins, or alkylated styrenes. Polyisobutylene is a commonly used viscosity index improver. Another suitable viscosity index improver is polymethacrylate (copolymers of various chain length alkyl methacrylates, for example), some formulations of which also serve as pour point depressants. Other suitable viscosity index improvers include copolymers of ethylene and propylene, hydrogenated block copolymers of styrene and isoprene, and polyacrylates (copolymers of various chain length acrylates, for example). Specific examples include styrene-isoprene or styrene-butadiene based polymers of 50,000 to 200,000 molecular weight.

The amount of viscosity modifier may range from zero to 8 wt %, preferably zero to 4 wt %, more preferably zero to 2 wt % based on active ingredient and depending on the specific viscosity modifier used.

### Antioxidants

Typical anti-oxidant include phenolic anti-oxidants, aminic anti-oxidants and oil-soluble copper complexes.

The phenolic antioxidants include sulfurized and non-sulfurized phenolic antioxidants. The terms “phenolic type” or “phenolic antioxidant” used herein includes compounds having one or more than one hydroxyl group bound to an aromatic ring which may itself be mononuclear, e.g., benzyl, or poly-nuclear, e.g., naphthyl and spiro aromatic compounds. Thus “phenol type” includes phenol per se, catechol, resorcinol, hydroquinone, naphthol, etc., as well as alkyl or alkenyl and sulfurized alkyl or alkenyl derivatives thereof, and bisphenol type compounds including such bi-phenol compounds linked by alkylene bridges sulfuric bridges or oxygen bridges. Alkyl phenols include mono- and poly-alkyl or alkenyl phenols, the alkyl or alkenyl group containing from about 3-100 carbons, preferably 4 to 50 carbons and sulfurized derivatives thereof, the number of alkyl or alkenyl groups present in the aromatic ring ranging from 1 to up to the available unsatisfied valences of the aromatic ring remaining after counting the number of hydroxyl groups bound to the aromatic ring.

Generally, therefore, the phenolic anti-oxidant may be represented by the general formula:

(R)ₓ+Ar+(OH)_{y}
where Ar is selected from the group consisting of:

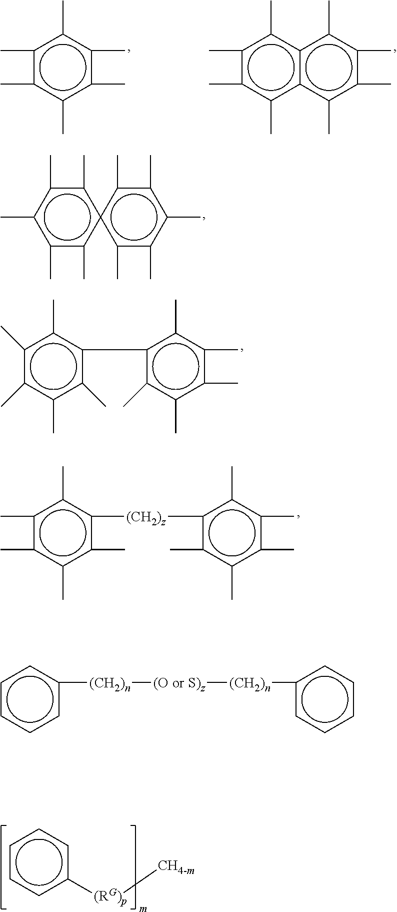

wherein R is a C₃-C₁₀₀ alkyl or alkenyl group, a sulfur substituted alkyl or alkenyl group, preferably a C₄-C₅₀ alkyl or alkenyl group or sulfur substituted alkyl or alkenyl group, more preferably C₃-C₁₀₀ alkyl or sulfur substituted alkyl group, most preferably a C₄-C₅₀ alkyl group, R^{G} is a C₁-C₁₀₀ alkylene or sulfur substituted alkylene group, preferably a C2-C₅₀ alkylene or sulfur substituted alkylene group, more preferably a C₂-C₂ alkylene or sulfur substituted alkylene group, y is at least 1 to up to the available valences of Ar, x ranges from 0 to up to the available valances of Ar—y, z ranges from 1 to 10, n ranges from 0 to 20, and m is 0 to 4 and p is 0 or 1, preferably y ranges from 1 to 3, x ranges from 0 to 3, z ranges from 1 to 4 and n ranges from 0 to 5, and p is 0.

Preferred phenolic anti-oxidant compounds are the hindered phenolics and phenolic esters which contain a sterically hindered hydroxyl group, and these include those derivatives of dihydroxy aryl compounds in which the hydroxyl groups are in the o- or p-position to each other. Typical phenolic anti-oxidants include the hindered phenols substituted with C₁+ alkyl groups and the alkylene coupled derivatives of these hindered phenols. Examples of phenolic materials of this type 2-t-butyl-4-heptyl phenol; 2-t-butyl-4-octyl phenol; 2-t-butyl-4-dodecyl phenol; 2,6-di-t-butyl-4-heptyl phenol; 2,6-di-t-butyl-4-dodecyl phenol; 2-methyl-6-t-butyl-4-heptyl phenol; 2-methyl-6-t-butyl-4-dodecyl phenol; 2,6-di-t-butyl-4 methyl phenol; 2,6-di-t-butyl-4-ethyl phenol; and 2,6-di-t-butyl 4 alkoxy phenol; and

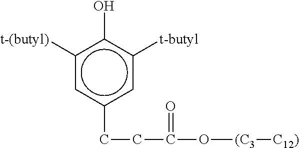

Phenolic type anti-oxidants are well known in the lubricating industry and commercial examples such as Ethanox® 4710, Irganox® 1076, Irganox® L1035, Irganox® 1010, Irganox® L109, Irganox® L118, Irganox® L135 and the like are familiar to those skilled in the art. The above is presented only by way of exemplification, not limitation on the type of phenolic anti-oxidants which can be used.

The phenolic anti-oxidant can be employed in an amount in the range of about 0.1 to 3 wt %, preferably about 1 to 3 wt %, more preferably 1.5 to 3 wt % on an active ingredient basis.

Aromatic amine anti-oxidants include phenyl-α-naphthyl amine which is described by the following molecular structure:

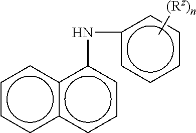

wherein R^{z} is hydrogen or a C₁ to C₁₄ linear or C₃ to C₁₄ branched alkyl group, preferably C₁ to C₁₀ linear or C₃ to C₁₀ branched alkyl group, more preferably linear or branched C₆ to C₈ and n is an integer ranging from 1 to 5 preferably 1. A particular example is Irganox L06.

Other aromatic amine anti-oxidants include other alkylated and non-alkylated aromatic amines such as aromatic monoamines of the formula R⁸R⁹R¹⁰N where R⁸ is an aliphatic, aromatic or substituted aromatic group, R⁹ is an aromatic or a substituted aromatic group, and R¹⁰ is H, alkyl, aryl or R¹¹S(O)ₓR¹² where R¹¹ is an alkylene, alkenylene, or aralkylene group, R¹² is a higher alkyl group, or an alkenyl, aryl, or alkaryl group, and x is 0, 1 or 2. The aliphatic group R⁸ may contain from 1 to about 20 carbon atoms, and preferably contains from about 6 to 12 carbon atoms. The aliphatic group is a saturated aliphatic group. Preferably, both R⁸ and R⁹ are aromatic or substituted aromatic groups, and the aromatic group may be a fused ring aromatic group such as naphthyl. Aromatic groups R⁸ and R⁹ may be joined together with other groups such as S.

Typical aromatic amines anti-oxidants have alkyl substituent groups of at least about 6 carbon atoms. Examples of aliphatic groups include hexyl, heptyl, octyl, nonyl, and decyl. Generally, the aliphatic groups will not contain more than about 14 carbon atoms. The general types of such other additional amine anti-oxidants which may be present include diphenylamines, phenothiazines, imidodibenzyls and diphenyl phenylene diamines. Mixtures of two or more of such other additional aromatic amines may also be present. Polymeric amine antioxidants can also be used.

Another class of anti-oxidant used in lubricating oil compositions and which may also be present are oil-soluble copper compounds. Any oil-soluble suitable copper compound may be blended into the lubricating oil. Examples of suitable copper antioxidants include copper dihydrocarbyl thio- or dithio-phosphates and copper salts of carboxylic acid (naturally occurring or synthetic). Other suitable copper salts include copper dithiacarbamates, sulphonates, phenates, and acetylacetonates. Basic, neutral, or acidic copper Cu(I) and or Cu(II) salts derived from alkenyl succinic acids or anhydrides are known to be particularly useful.

Such antioxidants may be used individually or as mixtures of one or more types of anti-oxidants, the total amount employed being an amount of about 0.50 to 5 wt %, preferably about 0.75 to 3 wt % (on an as-received basis).

### Detergents

In addition to the alkali or alkaline earth metal salicylate detergent which is an essential component in the present disclosure, other detergents may also be present. While such other detergents can be present, it is preferred that the amount employed be such as to not interfere with the synergistic effect attributable to the presence of the salicylate. Therefore, most preferably such other detergents are not employed.

If such additional detergents are present, they can include alkali and alkaline earth metal phenates, sulfonates, carboxylates, phosphonates and mixtures thereof. These supplemental detergents can have total base number (TBN) ranging from neutral to highly overbased, i.e. TBN of 0 to over 500, preferably 2 to 400, more preferably 5 to 300, and they can be present either individually or in combination with each other in an amount in the range of from 0 to 10 wt %, preferably 0.5 to 5 wt % (active ingredient) based on the total weight of the formulated lubricating oil. As previously stated, however, it is preferred that such other detergent not be present in the formulation.

Such additional other detergents include by way of example and not limitation calcium phenates, calcium sulfonates, magnesium phenates, magnesium sulfonates and other related components (including borated detergents).

### Dispersants

During engine operation, oil-insoluble oxidation byproducts are produced. Dispersants help keep these byproducts in solution, thus diminishing their deposition on metal surfaces. Dispersants may be ashless or ash-forming in nature. Preferably, the dispersant is ashless. So called ashless dispersants are organic materials that form substantially no ash upon combustion. For example, non-metal-containing or borated metal-free dispersants are considered ashless. In contrast, metal-containing detergents discussed above form ash upon combustion.

Suitable dispersants typically contain a polar group attached to a relatively high molecular weight hydrocarbon chain. The polar group typically contains at least one element of nitrogen, oxygen, or phosphorus. Typical hydrocarbon chains contain 50 to 400 carbon atoms.

A particularly useful class of dispersants are the alkenylsuccinic derivatives, typically produced by the reaction of a long chain substituted alkenyl succinic compound, usually a substituted succinic anhydride, with a polyhydroxy or polyamino compound. The long chain group constituting the oleophilic portion of the molecule which confers solubility in the oil, is normally a polyisobutylene group. Many examples of this type of dispersant are well known commercially and in the literature. Exemplary patents describing such dispersants are U.S. Pat. Nos. 3,172,892; 3,219,666; 3,316,177 and 4,234,435. Other types of dispersants are described in U.S. Pat. Nos. 3,036,003; and 5,705,458.

Hydrocarbyl-substituted succinic acid compounds are popular dispersants. In particular, succinimide, succinate esters, or succinate ester amides prepared by the reaction of a hydrocarbon-substituted succinic acid compound preferably having at least 50 carbon atoms in the hydrocarbon substituent, with at least one equivalent of an alkylene amine are particularly useful.

Succinimides are formed by the condensation reaction between alkenyl succinic anhydrides and amines. Molar ratios can vary depending on the amine or polyamine. For example, the molar ratio of alkenyl succinic anhydride to TEPA can vary from about 1:1 to about 5:1.

Succinate esters are formed by the condensation reaction between alkenyl succinic anhydrides and alcohols or polyols. Molar ratios can vary depending on the alcohol or polyol used. For example, the condensation product of an alkenyl succinic anhydride and pentaerythritol is a useful dispersant.

Succinate ester amides are formed by condensation reaction between alkenyl succinic anhydrides and alkanol amines. For example, suitable alkanol amines include ethoxylated polyalkylpolyamines, propoxylated polyalkylpolyamines and polyalkenylpolyamines such as polyethylene polyamines. One example is propoxylated hexamethylenediamine.

The molecular weight of the alkenyl succinic anhydrides will typically range between 800 and 2,500. The above products can be post-reacted with various reagents such as sulfur, oxygen, formaldehyde, carboxylic acids such as oleic acid, and boron compounds such as borate esters or highly borated dispersants. The dispersants can be borated with from about 0.1 to about 5 moles of boron per mole of dispersant reaction product.

Mannich base dispersants are made from the reaction of alkylphenols, formaldehyde, and amines. Process aids and catalysts, such as oleic acid and sulfonic acids, can also be part of the reaction mixture. Molecular weights of the alkylphenols range from 800 to 2,500.

Typical high molecular weight aliphatic acid modified Mannich condensation products can be prepared from high molecular weight alkyl-substituted hydroxyaromatics or HN(R)₂ group-containing reactants.

Examples of high molecular weight alkyl-substituted hydroxyaromatic compounds are polypropylphenol, polybutylphenol, and other polyalkylphenols. These polyalkylphenols can be obtained by the alkylation, in the presence of an alkylating catalyst, such as BF₃, of phenol with high molecular weight polypropylene, polybutylene, and other polyalkylene compounds to give alkyl substituents on the benzene ring of phenol having an average 600-100,000 molecular weight.

Examples of HN(R)₂ group-containing reactants are alkylene polyamines, principally polyethylene polyamines. Other representative organic compounds containing at least one HN(R)₂ group suitable for use in the preparation of Mannich condensation products are well known and include the mono- and di-amino alkanes and their substituted analogs, e.g., ethylamine and diethanol amine; aromatic diamines, e.g., phenylene diamine, diamino naphthalenes; heterocyclic amines, e.g., morpholine, pyrrole, pyrrolidine, imidazole, imidazolidine, and piperidine; melamine and their substituted analogs.

Examples of alkylene polyamine reactants include ethylenediamine, diethylene triamine, triethylene tetraamine, tetraethylene pentaamine, pentaethylene hexamine, hexaethylene heptaamine, heptaethylene octaamine, octaethylene nonaamine, nonaethylene decamine, and decaethylene undecamine and mixture of such amines having nitrogen contents corresponding to the alkylene polyamines, in the formula H₂N—(Z—NH—)ₙH, mentioned before, Z is a divalent ethylene and n is 1 to 10 of the foregoing formula. Corresponding propylene polyamines such as propylene diamine and di-, tri-, tetra-, pentapropylene tri-, tetra-, penta- and hexaamines are also suitable reactants. The alkylene polyamines are usually obtained by the reaction of ammonia and dihalo alkanes, such as dichloro alkanes. Thus the alkylene polyamines obtained from the reaction of 2 to 11 moles of ammonia with 1 to 10 moles of dichloroalkanes having 2 to 6 carbon atoms and the chlorines on different carbons are suitable alkylene polyamine reactants.

Aldehyde reactants useful in the preparation of the high molecular products useful in this disclosure include the aliphatic aldehydes such as formaldehyde (also as paraformaldehyde and formalin), acetaldehyde and aldol (β-hydroxybutyraldehyde). Formaldehyde or a formaldehyde-yielding reactant is preferred.

Preferred dispersants include borated and non-borated succinimides, including those derivatives from mono-succinimides, bis-succinimides, and/or mixtures of mono- and bis-succinimides, wherein the hydrocarbyl succinimide is derived from a hydrocarbylene group such as polyisobutylene having a Mn of from about 500 to about 5000 or a mixture of such hydrocarbylene groups. Other preferred dispersants include succinic acid-esters and amides, alkylphenol-polyamine-coupled Mannich adducts, their capped derivatives, and other related components. Such additives may be used in an amount of about 0.1 to 20 wt %, preferably about 0.1 to 8 wt %, more preferably about 1 to 6 wt % (on an as-received basis) based on the weight of the total lubricant.

### Pour Point Depressants

Conventional pour point depressants (also known as lube oil flow improvers) may also be present. Pour point depressant may be added to lower the minimum temperature at which the fluid will flow or can be poured. Examples of suitable pour point depressants include alkylated naphthalenes polymethacrylates, polyacrylates, polyarylamides, condensation products of haloparaffin waxes and aromatic compounds, vinyl carboxylate polymers, and terpolymers of dialkylfumarates, vinyl esters of fatty acids and allyl vinyl ethers. Such additives may be used in amount of about 0.0 to 0.5 wt %, preferably about 0 to 0.3 wt %, more preferably about 0.001 to 0.1 wt % on an as-received basis.

### Corrosion Inhibitors/Metal Deactivators

Corrosion inhibitors are used to reduce the degradation of metallic parts that are in contact with the lubricating oil composition. Suitable corrosion inhibitors include aryl thiazines, alkyl substituted dimercapto thiodiazoles thiadiazoles and mixtures thereof. Such additives may be used in an amount of about 0.01 to 5 wt %, preferably about 0.01 to 1.5 wt %, more preferably about 0.01 to 0.2 wt %, still more preferably about 0.01 to 0.1 wt % (on an as-received basis) based on the total weight of the lubricating oil composition.

### Seal Compatibility Additives

Seal compatibility agents help to swell elastomeric seals by causing a chemical reaction in the fluid or physical change in the elastomer. Suitable seal compatibility agents for lubricating oils include organic phosphates, aromatic esters, aromatic hydrocarbons, esters (butylbenzyl phthalate, for example), and polybutenyl succinic anhydride and sulfolane-type seal swell agents such as Lubrizol 730-type seal swell additives. Such additives may be used in an amount of about 0.01 to 3 wt %, preferably about 0.01 to 2 wt % on an as-received basis.

### Anti-Foam Agents

Anti-foam agents may advantageously be added to lubricant compositions. These agents retard the formation of stable foams. Silicones and organic polymers are typical anti-foam agents. For example, polysiloxanes, such as silicon oil or polydimethyl siloxane, provide antifoam properties. Anti-foam agents are commercially available and may be used in conventional minor amounts along with other additives such as demulsifiers; usually the amount of these additives combined is less than 1 percent, preferably 0.001 to about 0.5 wt %, more preferably about 0.001 to about 0.2 wt %, still more preferably about 0.0001 to 0.15 wt % (on an as-received basis) based on the total weight of the lubricating oil composition.

### Inhibitors and Antirust Additives

Anti-rust additives (or corrosion inhibitors) are additives that protect lubricated metal surfaces against chemical attack by water or other contaminants. One type of anti-rust additive is a polar compound that wets the metal surface preferentially, protecting it with a film of oil. Another type of anti-rust additive absorbs water by incorporating it in a water-in-oil emulsion so that only the oil touches the surface. Yet another type of anti-rust additive chemically adheres to the metal to produce a non-reactive surface. Examples of suitable additives include zinc dithiophosphates, metal phenolates, basic metal sulfonates, fatty acids and amines. Such additives may be used in an amount of about 0.01 to 5 wt %, preferably about 0.01 to 1.5 wt % on an as-received basis.

In addition to the ZDDP anti-wear additives which are essential components of the present disclosure, other anti-wear additives can be present, including zinc dithiocarbamates, molybdenum dialkyldithiophosphates, molybdenum dithiocarbamates, other organo molybdenum-nitrogen complexes, sulfurized olefins, etc.

The term “organo molybdenum-nitrogen complexes” embraces the organo molybdenum-nitrogen complexes described in U.S. Pat. 4,889,647. The complexes are reaction products of a fatty oil, dithanolamine and a molybdenum source. Specific chemical structures have not been assigned to the complexes. U.S. Pat. 4,889,647 reports an infrared spectrum for a typical reaction product of that disclosure; the spectrum identifies an ester carbonyl band at 1740 cm⁻¹ and an amide carbonyl band at 1620 cm⁻¹. The fatty oils are glyceryl esters of higher fatty acids containing at least 12 carbon atoms up to 22 carbon atoms or more. The molybdenum source is an oxygen-containing compound such as ammonium molybdates, molybdenum oxides and mixtures.

Other organo molybdenum complexes which can be used in the present disclosure are tri-nuclear molybdenum-sulfur compounds described in EP 1 040 115 and WO 99/31113 and the molybdenum complexes described in U.S. Pat. 4,978,464.

In the above detailed description, the specific embodiments of this disclosure have been described in connection with its preferred embodiments. However, to the extent that the above description is specific to a particular embodiment or a particular use of this disclosure, this is intended to be illustrative only and merely provides a concise description of the exemplary embodiments. Accordingly, the disclosure is not limited to the specific embodiments described above, but rather, the disclosure includes all alternatives, modifications, and equivalents falling within the true scope of the appended claims. Various modifications and variations of this disclosure will be obvious to a worker skilled in the art and it is to be understood that such modifications and variations are to be included within the purview of this application and the spirit and scope of the claims.

### EXAMPLES

### Example 1

### Synthesis of Alkylated Phenothiazine Using F24 Catalyst

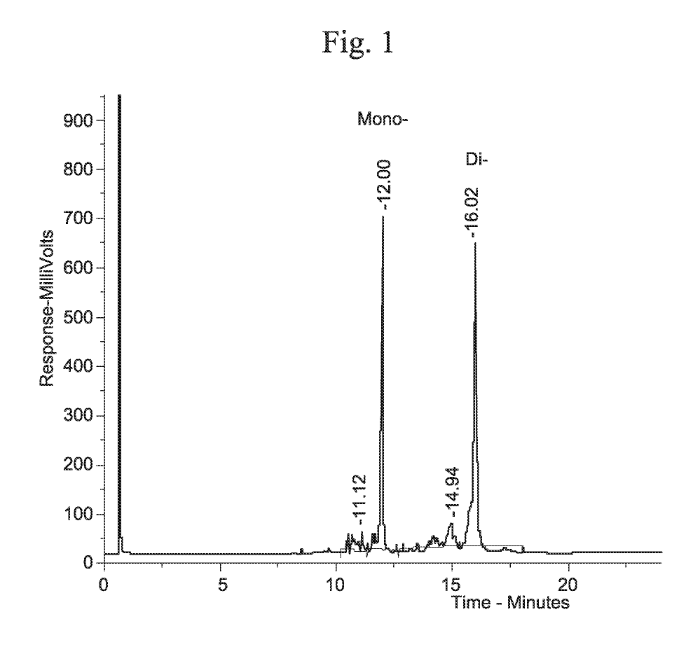

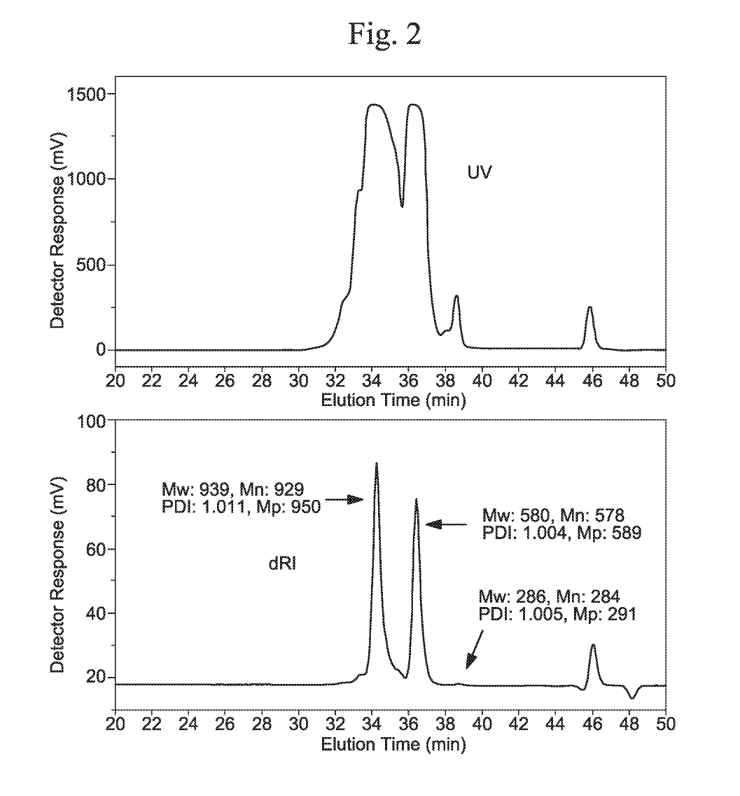

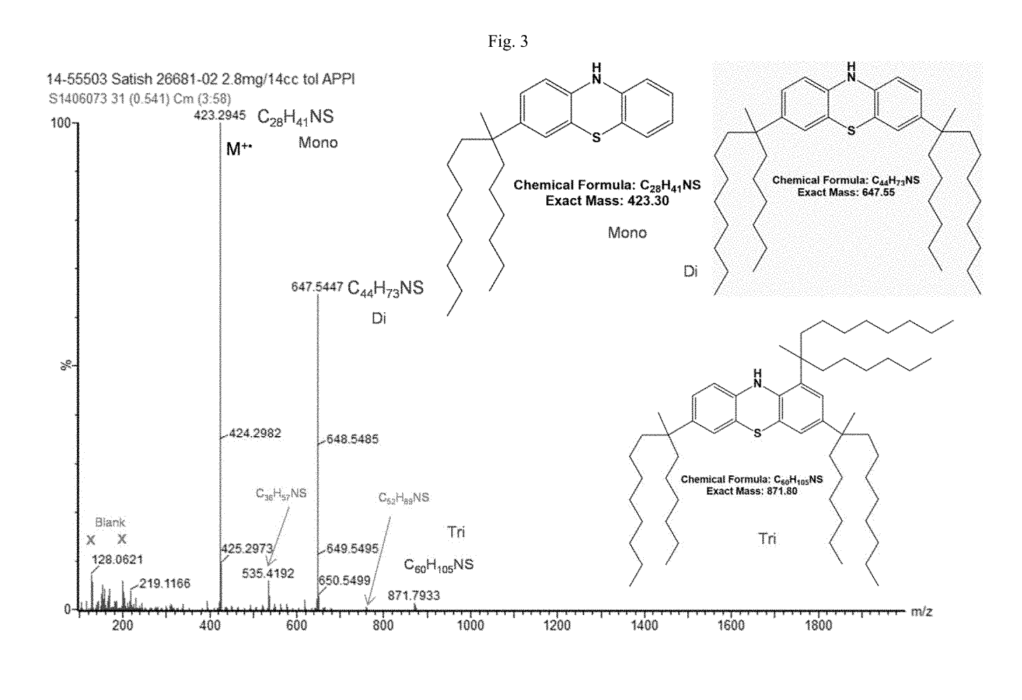

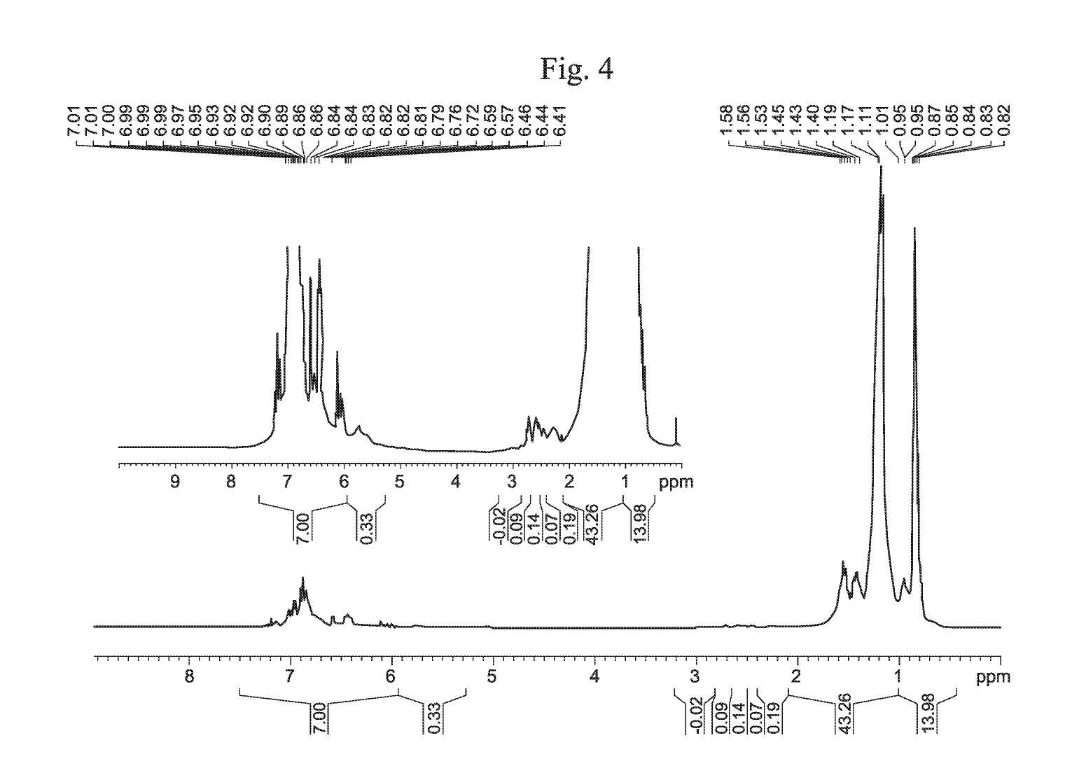

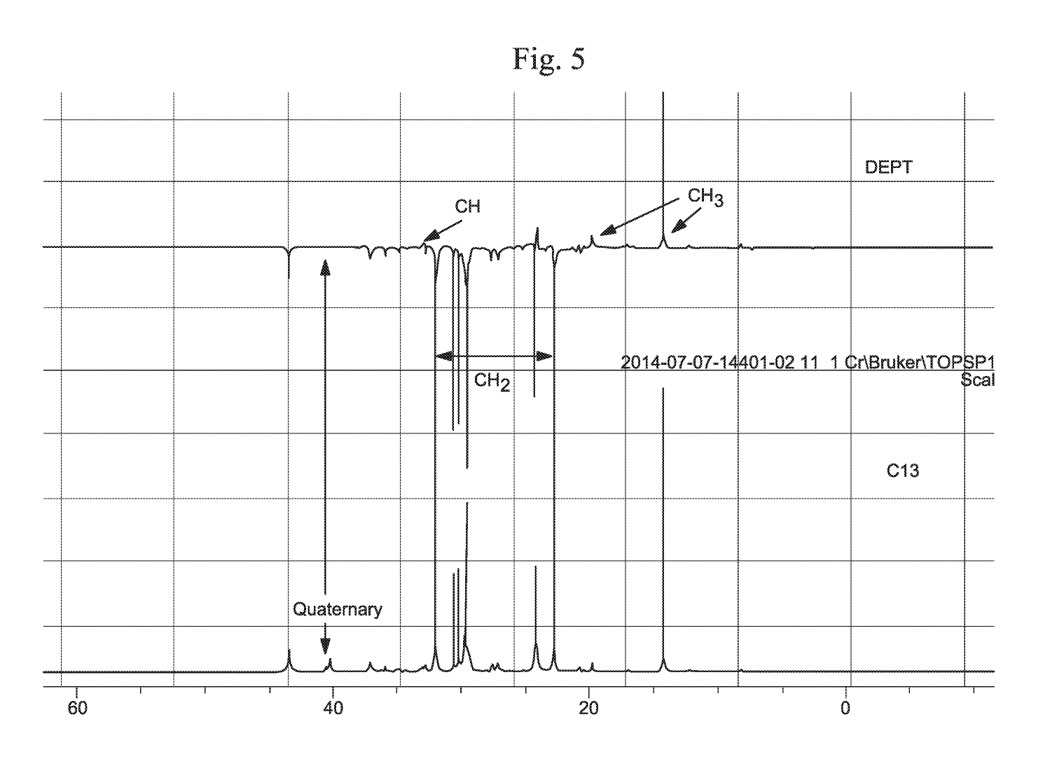

3.1 g Phenothiazine, 7.0 g mPAO-dimer (C₁₆═) and 2.0 g Engelhard F-24 (Filtrol 24; acid activated clay) were placed in 100 ml thick glass reactor. The reaction mixture was heated at 120° C. for 24 h under N₂. Toluene was added to dissolve the product and filtration was done to remove F24 catalyst. Unreacted dimer was removed by vacuum distillation at 200° C. Phenothiazine can be completely removed by flash chromatography with hexanes. Yield 5.5 g. ¹H-NMR and ¹³C-NMR spectrum showed disappearance of double bond and alkylation product. GC analysis of phenothiazine alkylated with mPAO-dimer (C₁₆═) using F24 catalyst. FIG. 1 shows gas chromatographic (GC) analysis suggesting that the product contain both mono- and di-alkylated phenothiazine products. GPC analysis of phenothiazine alkylated with mPAO-dimer (C₁₆═) using F24 catalyst. FIG. 2 shows gel permeation chromatography (GPC) analysis suggesting that the product contain both mono- and di-alkylated phenothiazine products. The GPC analysis was performed using both differential refractive index (DRI) and ultra violet (UV) detectors. FIG. 3 shows mass spectrum (MS) of phenothiazine alkylated with mPAO-dimer (C₁₆═) using F24 catalyst. The MS of the product suggests that mono- and di-alkylated phenothiazine products were obtained. The MS was performed using atmospheric pressure photoionization (APPI). FIG. 4 shows the ¹H-NMR spectrum of phenothiazine alkylated with mPAO-dimer (C₁₆═) using F24 catalyst. FIG. 5 shows the ¹³C-NMR and distortionless enhancement by polarization transfer (DEPT) spectrum of phenothiazine alkylated with mPAO-dimer (C₁₆═) using F24 catalyst. The results showed 37% non-quaternary carbon present in the product.

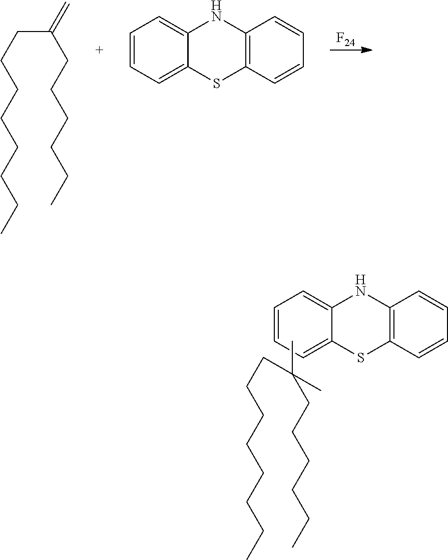

### Example 2

### Synthesis of Alkylated Phenothiazine Using MCM49 Catalyst

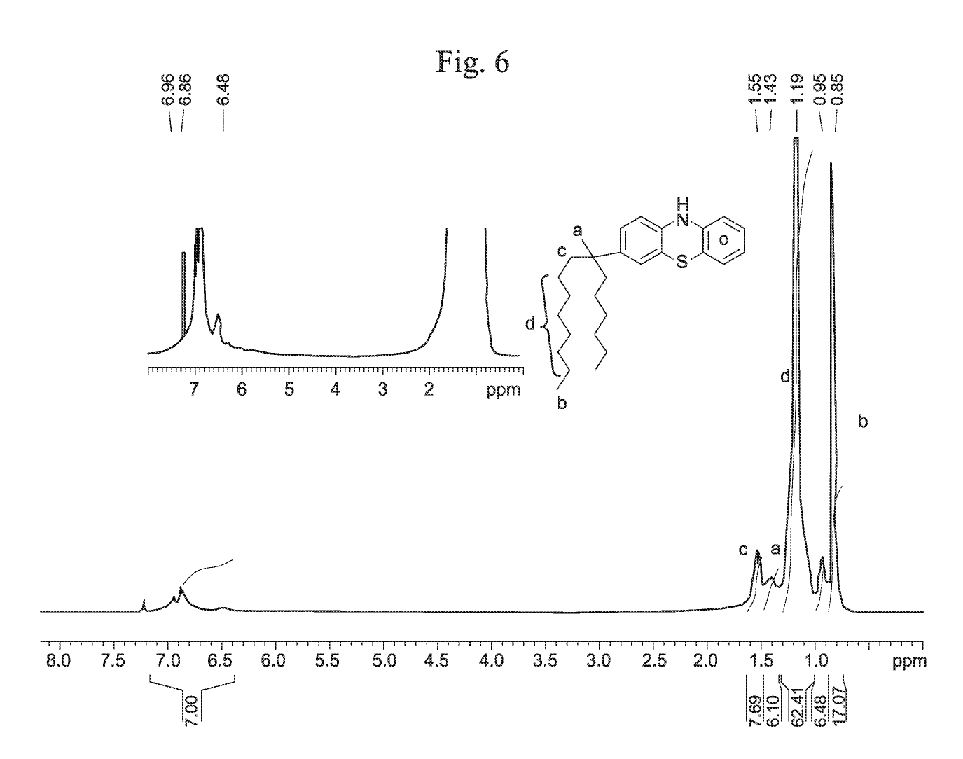

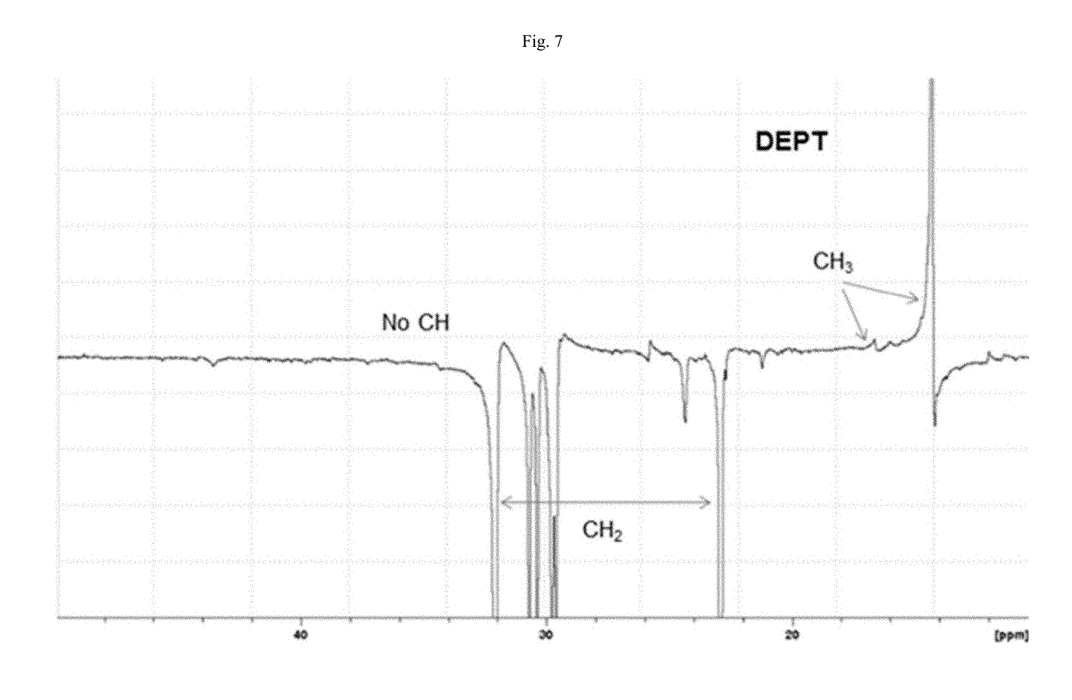

3.1 g Phenothiazine, 7.0 g mPAO-dimer (C₁₆═) and 2.0 g MCM-49 were charged in 100 ml thick glass reactor. The reaction mixture was heated at 150° C. for 24 h under N₂. Toluene was added to dissolve the product and filtration was done to remove MCM-49. Unreacted dimer was removed by vacuum distillation at 200° C. Phenothiazine can be completely removed by flash chromatography with hexanes. Yield 3.5 g. ¹H-NMR and ¹³C-NMR spectrum showed disappearance of double bond and alkylation product. FIG. 6 shows ¹H-NMR spectrum of phenothiazine alkylated with mPAO-dimer (C₁₆═) using MCM49 catalyst. The product was analyzed by GC, ¹H-NMR, ¹³C and DEPT. The results suggest that 11% mono-substitution and 88.6% di-substitution product was obtained based on GC and 100% quaternary carbon based on ¹H-NMR, ¹³C and DEPT results. FIG. 7 shows DEPT spectrum of phenothiazine alkylated with mPAO-dimer (C₁₆═) using MCM49 catalyst.

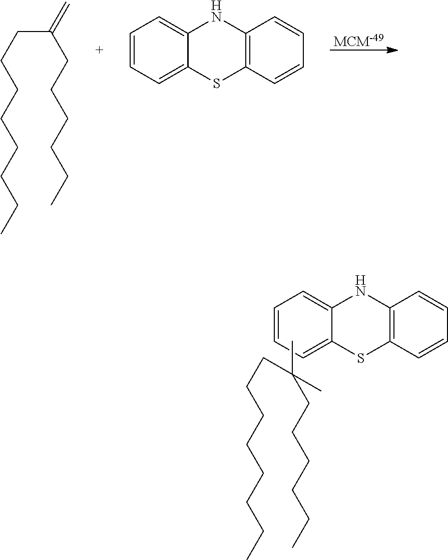

### Example 3

### Lube Properties and Oxidative Stability of Products

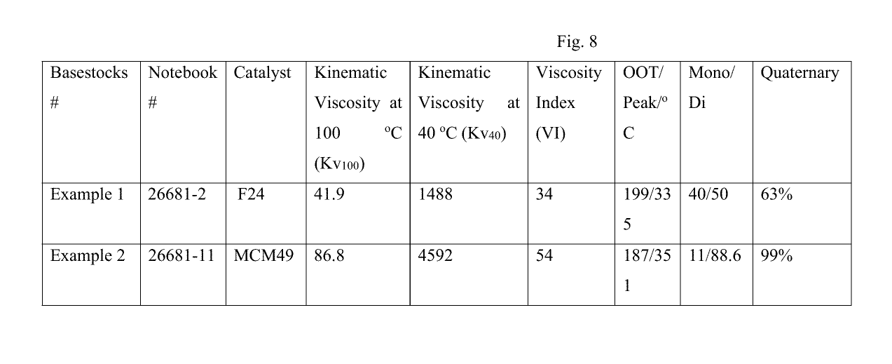

The lube properties and oxidative stability of the products of Examples 1 and 2 were evaluated and the data are shown in FIG. 8. The kinematic viscosity (Kv) of the liquid product was measured using ASTM standards D-445 and reported at temperatures of 100° C. (Kv at 100° C.) or 40° C. (Kv at 40° C.). The viscosity index (VI) was measured according to ASTM standard D-2270 using the measured kinematic viscosities for each product. The fluids were evaluated as synthetic base stocks and found to have good lubricant properties. The lube properties of the products of Examples 1 and 2 were evaluated and the data are shown in FIG. 8.

Phenothiazine was alkylated with 1-octene dimer using two catalysts (Engelhard F-24 (Filtrol 24; acid activated clay) and MCM 49 Zeolite catalyst). The MCM-49 catalyst gave a fluid with higher quaternary carbons than acid clay F24 catalyst. The alkylation chemistry converted non-lube byproduct into lube product.

### Example 4

### Synthesis of Alkylated Phenothiazine Using MCM49 Catalyst (Phenothiazine: mPAO-Dimer (C₁₆═)=3:1)

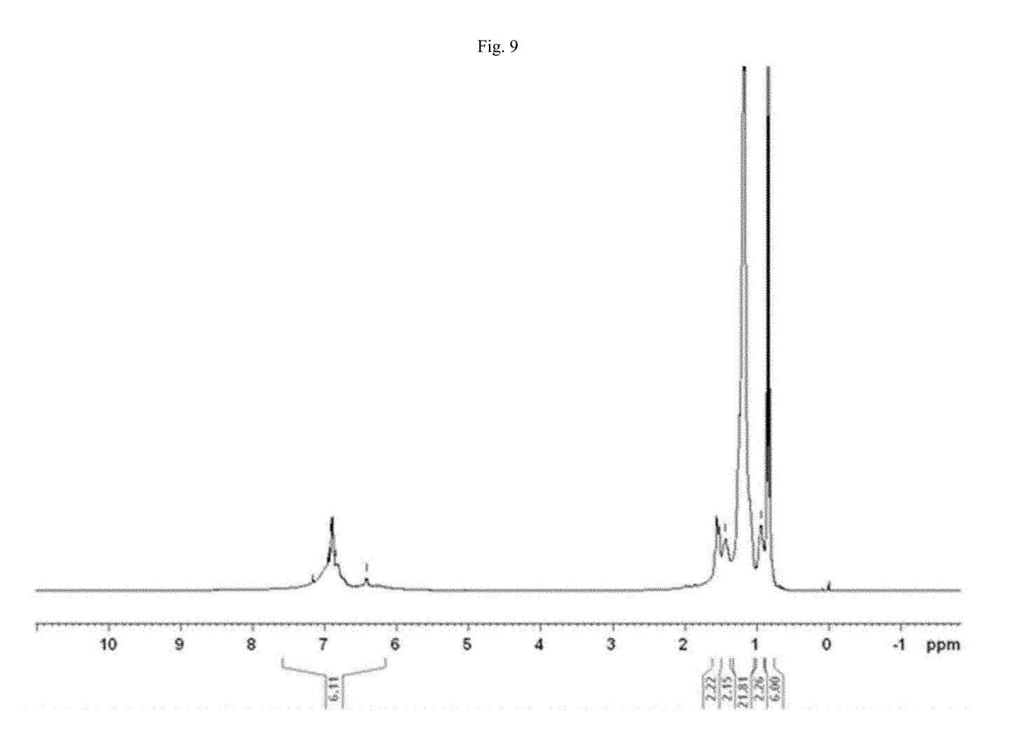

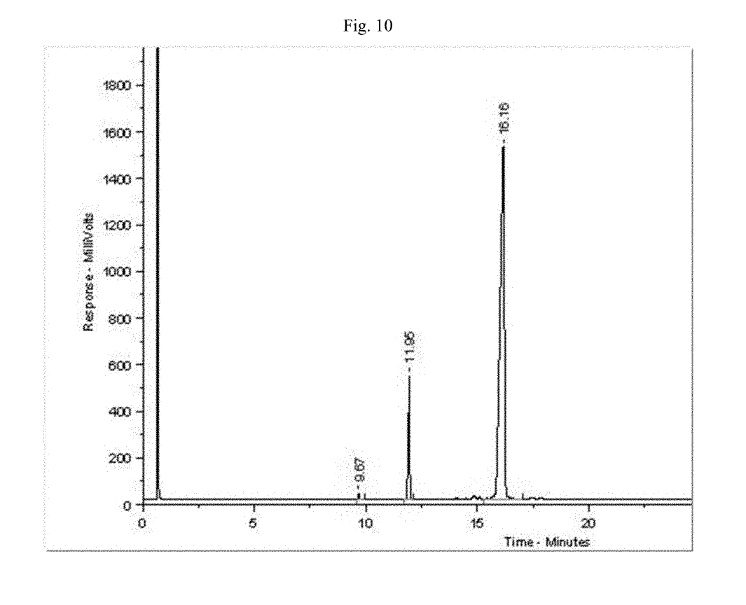

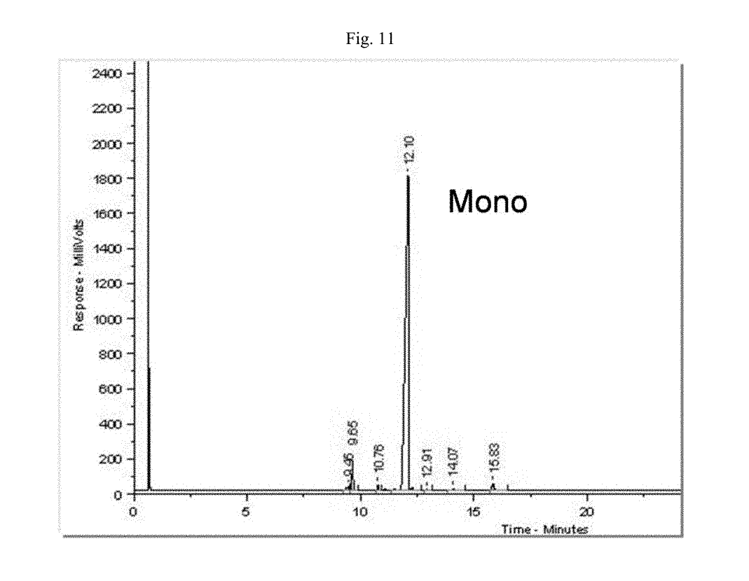

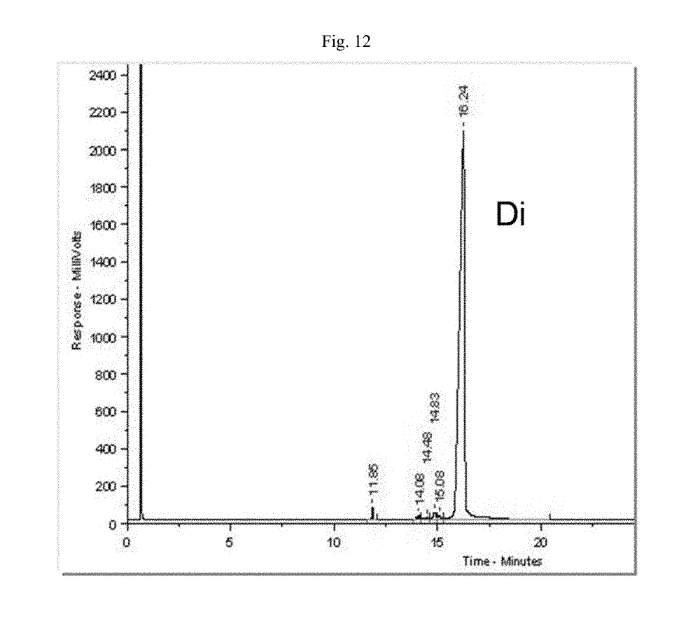

7.9 g Phenothiazine, 3.0 g mPAO-dimer (C₁₆═) and 4.0 g MCM49 in 100 ml thick glass reactor. The reaction was kept at 150° C. for 24 h. Toluene was added to dissolve the product and filtration was done to remove MCM49. Unreacted dimer was removed by vacuum distillation at 200° C. There was little product left in the bottle after vacuum distillation. FIG. 9 shows ¹H-NMR spectrum of alkylated phenothiazine prepared using MCM49 catalyst. Separation was performed of the phenothiazine alkylated mPAO-dimer (C₁₆═) prepared using MCM49 catalyst. FIG. 10 shows GC analysis of phenothiazine alkylated mPAO-dimer (C₁₆═) using MCM-96 catalyst product suggesting that both mono- and di-alkylated phenothiazine products were obtained. FIG. 11 shows GC analysis of fraction 1 of phenothiazine alkylated mPAO-dimer (C₁₆═) using MCM-96 catalyst. The GC analysis suggests that the mono-alkylated phenothiazine product was selectively fractionated. FIG. 12 shows GC analysis of fraction 2 of phenothiazine alkylated with mPAO-dimer (C₁₆═) using MCM-96 catalyst. The GC analysis suggests that the di-alkylated phenothiazine product was selectively fractionated.

The phenothiazine alkylated with 1-octene dimer (C₁₆═) using MCM-96 catalyst fluid containing both mono- and di-substituted alkylated product was successfully separated into individual products and their oxidation performance was evaluated using pressure differential scanning colorimetry (PDSC). The mixed product had oxidative onset temperature (OOT)/oxidation peak temperature (OPT) of 187° C./351° C. respectively. The separated mono-substituted product has OOT/OPT of 185° C./335° C. while di-substituted product had OOT/OPT of 202° C./365° C. The results suggest that di-substituted phenothiazine has better oxidative stability than mono-substituted product. For alkylation reaction, other olefins can be used, for example, (α-olefins, unhydrogenated poly-α-olefins , unhydrogenated ethylene a-olefin copolymers, terminal double bond containing macromers), phenothiazine derivatives such as phenyl phenothiazine and any other acidic catalysts like Lewis acid, Bronsted acids, acidic ionic liquids, and the like.

Pressure Differential Scanning calorimetry (PDSC) is a useful screening tool for measuring oxidative stability. PDSC is used to determine oxidation under heating conditions. A heating experiment measures the temperature at which oxidation initiates under oxygen pressure. A DSC Model 2920 (TA instruments) with a pressure cell was used for the measurements. The cell is well calibrated for temperature (+/−0.3° C.) and heat flow (better than 1%) and checked for reproducibility daily with a quality control standard for temperature and heat response. The heating measurements were carried out at a heating rate of 10° C./min using pressure of 100 psi in air.

### Example 5

Phenothiazine was alkylated with C16 mPAO dimer (vinylidene/trisubsitituted olefin) by catalytic p-toluenesulfonic acid monohydrate in toluene at 120° C. The lube product consisted of monoalkylated and dialkylated phenothiazine. GC evidence showed that only one isomer each of the monoalkylated and dialkylated products were formed.

There was no observed conversion when phenothiazine was alkylated with a C14 LAO under otherwise identical conditions.

There was no observed conversion when phenothiazine was reacted with C16 mPAO dimer and USY-type zeolite catalysts with reaction temperatures up to 210° C.

Phenothiazine was alkylated with LAOs (C8, C10, C12) by catalytic triflic acid in decane at 150° C. The lube product consisted of multiple isomers each of mono-, di-, trialkylated phenothiazine.

Alkylated phenothiazine formulated at 5 wt. % in SpectraSyn™ 4 PAO significantly improved the oxidative stability compared to neat SpectraSyn™ 4 PAO in a rotating pressure vessel oxidation test (RPVOT) according to ASTM D2272.

### Example 6

### Phenothiazine Alkylated with mPAO Dimer (p-TsOH Catalyst)

A round bottom flask equipped with a Dean-Stark trap was charged with p-toluenesulfonic acid monohydrate (2.39 g, 12.6 mmol, 5 mol %) and toluene (200 mL). The mixture was refluxed for 30 minutes then cooled to 80° C. Phenothiazine (50.01 g, 0.25 mol) was added and the colorless solution instantly turns bright red. The temperature is increased to 90° C. and C16 mPAO dimer (56.50 g, 0.25 mol, 1.0 equiv.) was added drop wise over 30 minutes. The temperature was slowly increased to 120° C. over 3 hours. The reaction continued at 120° C. for 24 hours and then was allowed to cool to room temperature. The reaction mixture was diluted with hexanes (200 mL) and filtered to remove most of the unreacted phenothiazine. The filtrate was distilled at 130° C. under N₂ atmosphere to remove toluene and hexanes. The material was then distilled under oil pump vacuum to 160° C. to remove unreacted olefin and phenothiazine. The resulting deep violet oil was transferred to a storage container and cooled to room temperature (lube yield 56.15 g).

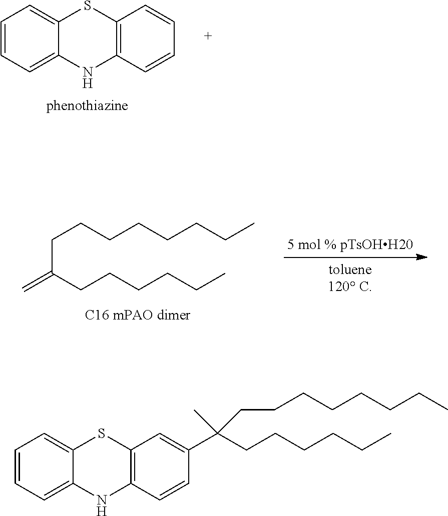

### Example 7

### Phenothiazine Alkylated with LAO (Triflic Acid Catalyst)

A glass reactor was charged with phenothiazine (750 g, 3.7 mol), 1-dodecene (1268 g, 7.4 mol, 2.0 equiv.), decane (300 g), and triflic acid (5 mL, 56.5 mmol, 1.5 mol %). The mixture was heated to 160° C. with stirring and allowed to react for 24 hours. The temperature was decreased to 100° C. and NaHCO₃ (25 g) was added. The mixture was stirred for 1 hour and filtered. The filtrate was distilled (2 Torr, 190° C.) to remove decane, dodecene, and unreacted phenothiazine. The green oil was transferred to a storage container and cooled to room temperature (lube yield 1375 g).

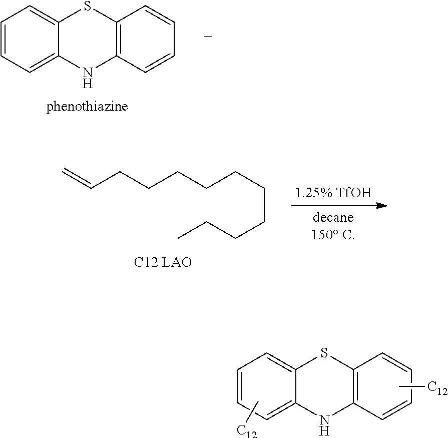

### Lube Properties and Oxidative Stability of Products

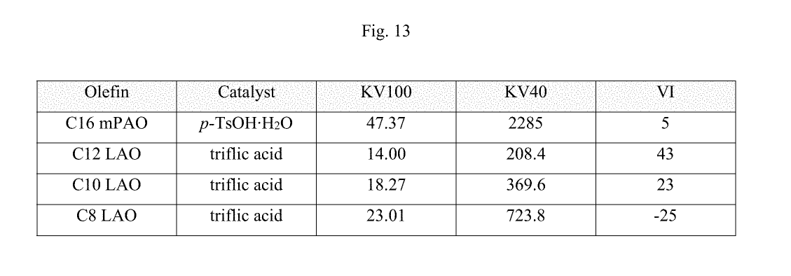

The lube properties and oxidative stability of the products of Examples 6 and 7 were evaluated and the data are shown below. The kinematic viscosity (Kv) of the liquid product was measured using ASTM standards D-445 and reported at temperatures of 100° C. (Kv at 100° C.) or 40° C. (Kv at 40° C.). The viscosity index (VI) was measured according to ASTM standard D-2270 using the measured kinematic viscosities for each product. The fluids were evaluated as synthetic base stocks and found to have good lubricant properties. The lube properties of the products of Examples 6 and 7 were evaluated and the data are shown in FIG. 13.

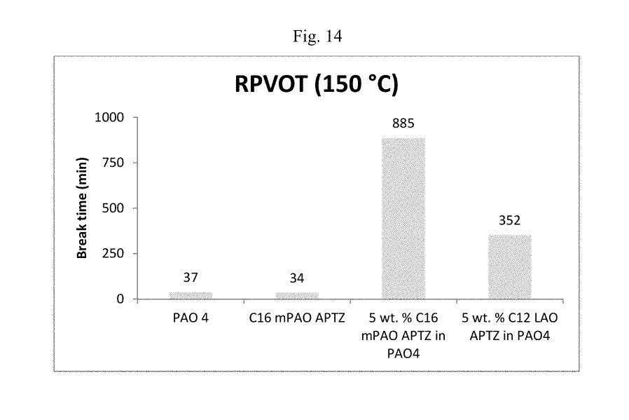

C16 mPAO APTZ and C12 LAO APTZ were each blended at 5 wt. % in SpectraSyn™ 4 PAO. The oxidative stability of the two blends was measured by RPVOT according to ASTM D2272. As shown in FIG. 14, the results are compared against the RPVOT performances of neat SpectraSyn™ 4 PAO and neat C16 mPAO APTZ.

As a neat base stock, C16 mPAO APTZ performs similar to SpectraSyn™ 4 PAO in the RPVOT test. However, at lower concentrations, C16 mPAO APTZ displays an antioxidant effect in SpectraSyn™ 4 PAO by increasing the RPVOT break time from 37 minutes to 885 minutes. C12 LAO APTZ displayed a similar, albeit smaller antioxidant effect by increasing the RPVOT break time to 352 minutes.

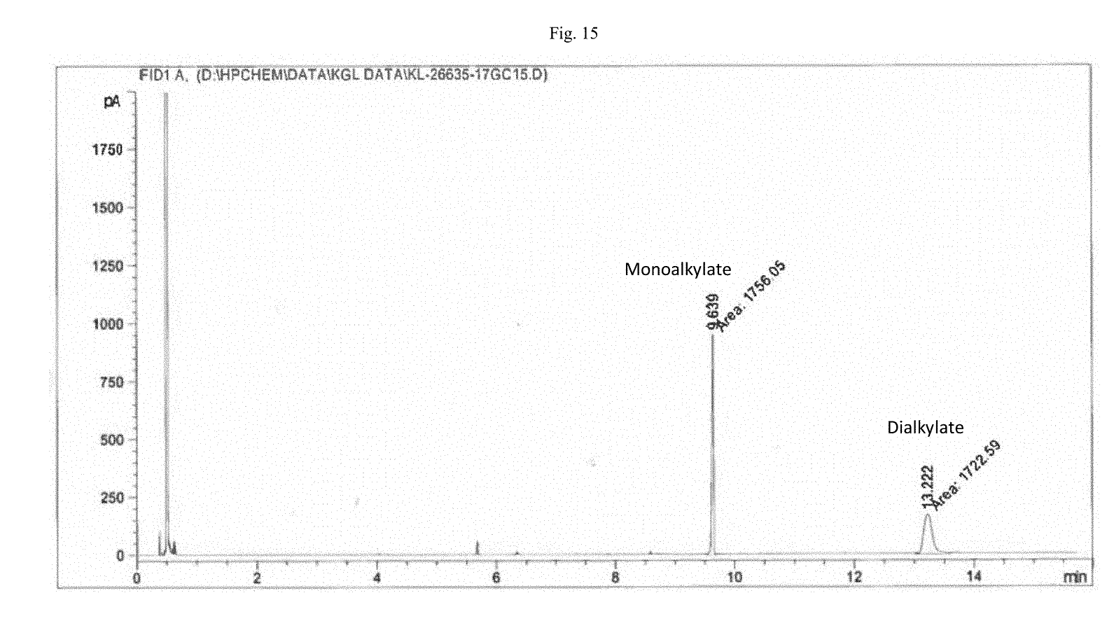

A GC analysis of C16 mPAO APTZ (p-TsOH catalyst) was conducted. FIG. 15 shows GC analysis suggesting that the product contains both mono- and di-alkylated phenothiazine.

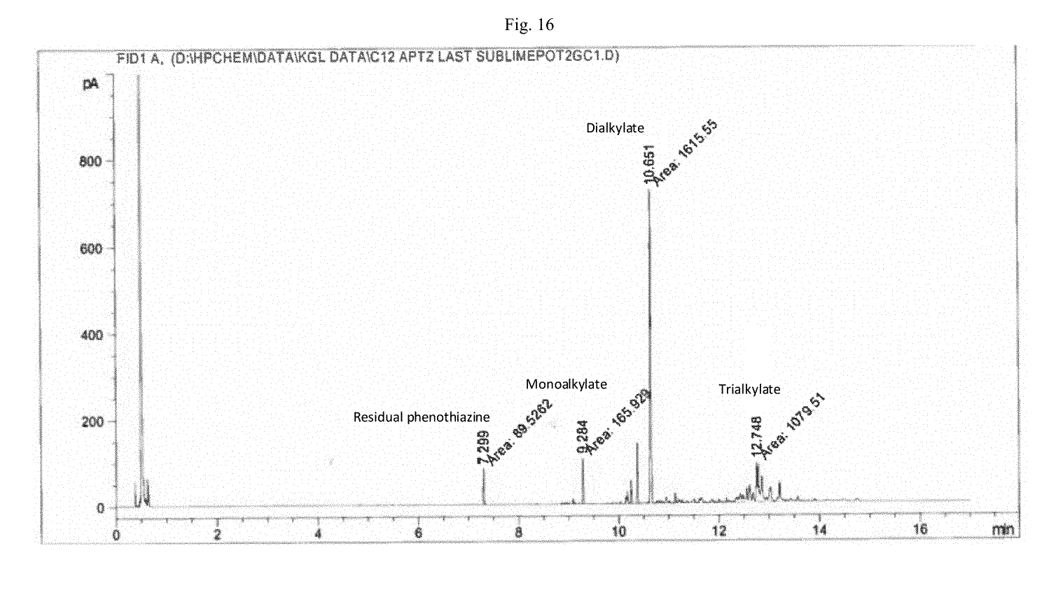

A GC analysis of C12 LAO APTZ (triflic acid catalyst) was conducted. FIG. 16 shows GC analysis suggesting that the product contains predominantly dialkylated phenothiazine along with smaller amounts of mono- and trialkylated phenothiazine. Less than 4% residual phenothiazine was present.

### Example 8

In an embodiment, this disclosure relates to the synthesis of mPAO dimer (C₁₆═, C₂₀═) alkylated with phenothiazine. Phenothiazine acts as both primary (free radical) and secondary (hydroperoxide decomposer) inhibitor and has lower bond dissociation energy than diphenylamine. The products exhibit good lubricant properties. An illustrative reaction is as follows:

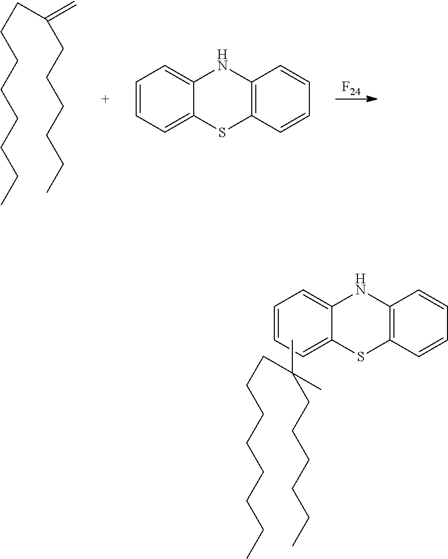

Phenothiazine was alkylated with 1-octene dimer (C₁₆═) using two catalysts (Engelhard F-24: Filtrol 24; acid activated clay) and MCM 49 (zeolite catalyst). The alkylation process produced a mixture predominantly of mono and dialkylated compounds with only trace amounts of higher alkylated materials being formed. However, both catalysts gave different percent of quaternary carbons in alkylated products. The MCM-49 catalyst gave fluid with higher quaternary carbons than acid clay F24 catalyst. The F24 catalyst based product had kinematic viscosity at 100° C. (Kv₁₀₀) of 41.9 cSt with Viscosity Index (VI) of 34, while the MCM-49 catalyst based product had a Kv₁₀₀ of 86.8 cSt with VI of 54. Thus, the alkylation chemistry of this disclosure converted non-lube byproducts into lube products.

The phenothiazine alkylated with 1-octene dimer (C₁₆═) using MCM-96 catalyst fluid containing both mono- and di-alkylated product was successfully separated into individual products and their oxidation performance was evaluated using PDSC. The mixed product had oxidative onset temperature (OOT)/oxidation peak temperature (OPT) of 187/351° C. respectively. The separated mono-alkylated product had OOT/OPT of 185 /335° C. while di-alkylated product had OOT/OPT of 202/365° C. The results show that the di-alkyl substituted phenothiazine had better oxidative stability than mono- alkyl substituted product.

For alkylation reaction, illustrative olefins that can be used include, for example, α-olefins, internal olefins, unhydrogenated poly-α-olefins, unhydrogenated ethylene α-olefin copolymers, unhydrogenated polyisobutylene, olefins with terminal double bond containing macromers, and the like.

Besides phenothiazine, one can use phenothiazine derivatives such as 2-methylthiophenothiazine, 10-methylphenothiazine, monotetradecylphenothiazine, ditetradecylphenothiazine, monodecylphenothiazine, didecylphenothiazine, monononylphenothiazine, dinonylphenothiazine, monoctylphenothiazine, dioctylphenothiazine, monobutylphenothiazine, dibutylphenothiazine, monostyrylphenothiazine, distyrylphenothiazine, butyloctylphenothiazine, styryloctylphenothiazine, and mixtures thereof, and the like.

The alkylation catalyst can be selected from Bronsted acids, Lewis acids, aluminum silicates, ion exchange resins, zeolites, naturally occurring sheet silicates, modified sheet silicates and acidic ionic liquids, and the like.

Phenothiazine was also alkylated with C₁₆ mPAO dimer (vinylidene/trisubsitituted olefin) by catalytic p-toluenesulfonic acid monohydrate in toluene at 120° C. The lube product consisted of monoalkylated and dialkylated phenothiazine. As shown by GC evidence, only one isomer each of the monoalkylated and dialkylated products was formed. There was no observed conversion when phenothiazine was alkylated with a C₁₄ LAO under otherwise identical conditions. There was no observed conversion when phenothiazine was reacted with C₁₆ mPAO dimer and USY-type zeolite catalysts with reaction temperatures up to 210° C.

Phenothiazine was alkylated with LAOs (C₈, C₁₀, C₁₂) by catalytic triflic acid in decane at 150° C. The lube product consisted of multiple isomers each of mono-, di-, trialkylated phenothiazine.

Alkylated phenothiazine formulated at 5 wt. % in SpectraSyn™ 4 PAO significantly improves the oxidative stability compared to neat SpectraSyn™ 4 PAO (RPVOT). Thus, RPVOT data show that alkylated phenothiazine has good antioxidant properties at low blend concentration.

### PCT and EP Clauses:

1. A composition comprising one or more compounds represented by the formula

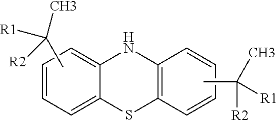

wherein R1 and R2 are same or different and are the residue of an alkyl group having more than 10 carbon atoms wherein the composition has a viscosity (Kv₁₀₀) from 2 to 300 at 100° C. as determined by ASTM D-445, and a viscosity index (VI) from −100 to 300 as determined by ASTM D-2270.

2. The composition of claim 1 wherein R₁ and R₂ are selected from the residue of a mPAO dimer (C₁₂-C₂₈), trimer (C₁₈-C₄₂), tetramer (C₂₄-C₅₆), pentamer (C₃₀-C₇₀), and hexamer (C₃₆-C₈₄).

3. The composition of clauses 1 and 2 wherein the polycyclic, heteroatom-containing, hydrocarbon compound comprises phenothiazine, a derivative of phenothiazine, 2-methylthiophenothiazine, 10-methylphenothiazine, monotetradecylphenothiazine, ditetradecylphenothiazine, monodecylphenothiazine, didecylphenothiazine, monononylphenothiazine, dinonylphenothiazine, monoctylphenothiazine, dioctylphenothiazine, monobutylphenothiazine, dibutylphenothiazine, monostyrylphenothiazine, distyrylphenothiazine, butyloctylphenothiazine, styryloctylphenothiazine, and mixtures thereof.

4. A composition comprising one or more alkylated polycyclic, heteroatom-containing, hydrocarbon compounds, wherein said one or more alkylated polycyclic, heteroatom-containing, hydrocarbon compounds are produced by a process comprising reacting a polyalphaolefin oligomer with at least one polycyclic, heteroatom-containing, hydrocarbon compound, in the presence of a catalyst, under reaction conditions sufficient to produce said one or more alkylated polycyclic, heteroatom-containing, hydrocarbon compounds.

5. The composition of clause 4 wherein the polycyclic, heteroatom-containing, hydrocarbon compound is represented by the formula

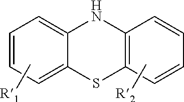

wherein R′₁ and R′₂ are the same or different and are hydrogen or the residue of an alkyl group having from 1 to 40 carbon atoms.

6. A process for producing a composition comprising one or more alkylated polycyclic, heteroatom-containing, hydrocarbon compounds, said process comprising reacting a polyalphaolefin oligomer with at least one polycyclic, heteroatom-containing, hydrocarbon compound, in the presence of a catalyst, under reaction conditions sufficient to produce said composition.

7. The process of clause 6 which is carried out under reaction conditions sufficient to couple the polyalphaolefin oligomer with the at least one polycyclic, heteroatom-containing, hydrocarbon compound, to produce said composition.

8. A lubricating oil base stock comprising one or more compounds represented by the formula

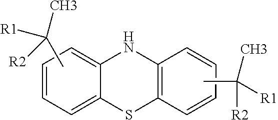

wherein R1 and R2 are same or different and are the residue of an alkyl group having more than 10 carbon atoms wherein the composition has a viscosity (Kv₁₀₀) from 2 to 300 at 100° C. as determined by ASTM D-445, and a viscosity index (VI) from −100 to 300 as determined by ASTM

D-2270.

9. The lubricating oil base stock of clause 8 wherein R₁ and R₂ are selected from the residue of a mPAO dimer (C₁₂-C₂₈), trimer (C₁₈-C₄₂), tetramer (C₂₄-C₅₆), pentamer (C₃₀-C₇₀), and hexamer (C₃₆-C₈₄).

10. The lubricating oil base stock of clauses 8 and 9 wherein the polycyclic, heteroatom-containing, hydrocarbon compound comprises phenothiazine, a derivative of phenothiazine, 2-m ethylthi ophenothi azine, 10-methylphenothiazine, monotetradecylphenothiazine, ditetradecylphenothiazine, monodecylphenothiazine, didecylphenothiazine, monononylphenothiazine, dinonylphenothiazine, monoctylphenothiazine, dioctylphenothiazine, monobutylphenothiazine, dibutylphenothiazine, monostyrylphenothiazine, distyrylphenothiazine, butyloctylphenothiazine, styryloctylphenothiazine, and mixtures thereof.

11. The lubricating oil base stock of clauses 8-10 which comprises an alkylated phenothiazine, an alkylated derivative of phenothiazine, or mixtures thereof.

12. A lubricating oil comprising a lubricating oil base stock as a major component, and a polycyclic, heteroatom-containing, hydrocarbon compound cobase stock as a minor component; wherein said polycyclic, heteroatom-containing, hydrocarbon compound cobase stock comprises one or more compounds represented by the formula

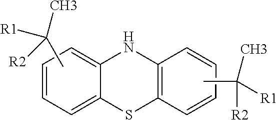

wherein R1 and R2 are same or different and are the residue of an alkyl group having more than 10 carbon atoms; wherein said polycyclic, heteroatom-containing, hydrocarbon compound cobase stock has a viscosity (Kv₁₀₀) from 2 to 300 at 100° C. as determined by ASTM D-445, and a viscosity index (VI) from −100 to 300 as determined by ASTM D-2270.

13. A method for improving oxidative stability of a lubricating oil by using as the lubricating oil a formulated oil comprising a lubricating oil base stock as a major component, and polycyclic, heteroatom-containing, hydrocarbon compound cobase stock as a minor component; wherein said polycyclic, heteroatom-containing, hydrocarbon compound cobase stock comprises one or more compounds represented by the formula

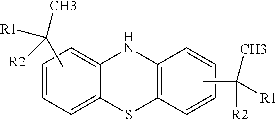

wherein R1 and R2 are same or different and are the residue of an alkyl group having more than 10 carbon atoms; wherein said polycyclic, heteroatom-containing, hydrocarbon compound cobase stock has a viscosity (Kv₁₀₀) from 2 to 300 at 100° C. as determined by ASTM D-445, a viscosity index (VI) from −100 to 300 as determined by ASTM D-2270, and an oxidation onset temperature (OOT)/oxidation peak temperature (OPT) ranging from 175° C./325° C. to 225° C./375° C.; wherein oxidative stability is improved as compared to oxidative stability achieved using a lubricating engine oil containing a minor component other than the polycyclic, heteroatom-containing, hydrocarbon compound cobase stock.

14. A composition comprising one or more compounds represented by the formula

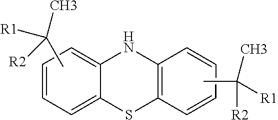

wherein R1 and R2 are same or different and are the residue of an alkyl group having more than 10 carbon atoms, wherein the composition has a viscosity (Kv₁₀₀) from 2 to 300 at 100° C. as determined by ASTM D-445, and a viscosity index (VI) from −100 to 300 as determined by ASTM D-2270.

15. A lubricating oil base stock comprising one or more compounds represented by the formula

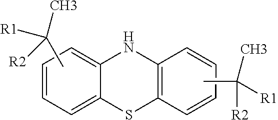

wherein R1 and R2 are same or different and are the residue of an alkyl group having more than 10 carbon atoms, wherein the composition has a viscosity (Kv₁₀₀) from 2 to 300 at 100° C. as determined by ASTM D-445, and a viscosity index (VI) from −100 to 300 as determined by ASTM D-2270.

All patents and patent applications, test procedures (such as ASTM methods, UL methods, and the like), and other documents cited herein are fully incorporated by reference to the extent such disclosure is not inconsistent with this disclosure and for all jurisdictions in which such incorporation is permitted.

When numerical lower limits and numerical upper limits are listed herein, ranges from any lower limit to any upper limit are contemplated. While the illustrative embodiments of the disclosure have been described with particularity, it will be understood that various other modifications will be apparent to and can be readily made by those skilled in the art without departing from the spirit and scope of the disclosure. Accordingly, it is not intended that the scope of the claims appended hereto be limited to the examples and descriptions set forth herein but rather that the claims be construed as encompassing all the features of patentable novelty which reside in the present disclosure, including all features which would be treated as equivalents thereof by those skilled in the art to which the disclosure pertains.

The present disclosure has been described above with reference to numerous embodiments and specific examples. Many variations will suggest themselves to those skilled in this art in light of the above detailed description. All such obvious variations are within the full intended scope of the appended claims.

1. A composition comprising one or more compounds represented by the formula 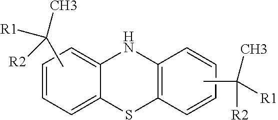
wherein R1 and R2 are same or different and are the residue of an alkyl group having more than 10 carbon atoms wherein the composition has a viscosity (Kv₁₀₀) from 2 to 300 at 100° C. as determined by ASTM D-445, and a viscosity index (VI) from −100 to 300 as determined by ASTM D-2270.

2. The composition of claim 1 wherein R₁ and R₂ are selected from the residue of a mPAO dimer (C₁₂-C₂₈), trimer (C₁₈-C₄₂), tetramer (C₂₄-C₅₆), pentamer (C₃₀-C₇₀), and hexamer (C₃₆-C₈₄).

3. The composition of claim 1 wherein the polycyclic, heteroatom-containing, hydrocarbon compound comprises phenothiazine, a derivative of phenothiazine, 2-methylthiophenothiazine, 10-methylphenothi azine, monotetradecylphenothiazine, ditetradecylphenothiazine, monodecylphenothiazine, didecylphenothiazine, monononylphenothiazine, dinonylphenothiazine, monoctylphenothiazine, dioctylphenothiazine, monobutylphenothiazine, dibutylphenothiazine, monostyrylphenothiazine, distyrylphenothiazine, butyloctylphenothiazine, styryloctylphenothiazine, and mixtures thereof.

4. The composition of claim 1 which comprises one or more alkylated polycyclic, heteroatom-containing, hydrocarbon compounds.

5. The composition of claim 3 which comprises an alkylated phenothiazine, an alkylated derivative of phenothiazine, or mixtures thereof.

6. The composition of claim 4 wherein the one or more alkylated polycyclic, heteroatom-containing, hydrocarbon compounds comprise monoalkylated polycyclic, heteroatom-containing, hydrocarbon compounds and dialkylated polycyclic, heteroatom-containing, hydrocarbon compounds, wherein the ratio of monoalkylated polycyclic, heteroatom-containing, hydrocarbon compounds to dialkylated polycyclic, heteroatom-containing, hydrocarbon compounds is from 5:95 to 95:5.

7. The composition of claim 4 wherein the alkylated polycyclic, heteroatom-containing, hydrocarbon compounds have a quaternary carbon content from 50 percent to 100 percent, based on the total primary, secondary, tertiary and quaternary carbons in the alkylated polycyclic, heteroatom-containing, hydrocarbon compounds.

8. The composition of claim 1 which is selected from a polycyclic, heteroatom-containing, hydrocarbon compound-containing mPAO dimer, trimer, tetramer, pentamer, hexamer and higher oligomer.

9. A composition comprising one or more alkylated polycyclic, heteroatom-containing, hydrocarbon compounds, wherein said one or more alkylated polycyclic, heteroatom-containing, hydrocarbon compounds are produced by a process comprising reacting a polyalphaolefin oligomer with at least one polycyclic, heteroatom-containing, hydrocarbon compound, in the presence of a catalyst, under reaction conditions sufficient to produce said one or more alkylated polycyclic, heteroatom-containing, hydrocarbon compounds.

10. The composition of claim 9 wherein the one or more alkylated polycyclic, heteroatom-containing, hydrocarbon compounds are represented by the formula 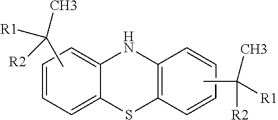
wherein R1 and R2 are same or different and are the residue of an alkyl group having more than 10 carbon atoms wherein the composition has a viscosity (Kv₁₀₀) from 2 to 300 at 100° C. as determined by ASTM D-445, and a viscosity index (VI) from −100 to 300 as determined by ASTM D-2270.

11. The composition of claim 10 wherein R₁ and R₂ are selected from the residue of a mPAO dimer (C₁₂-C₄₂), trimer (C₁₈-C₄₂), tetramer (C₂₄-₅₆, pentamer (C₃₀-C₇₀), and hexamer (C₃₆-C₈₄).

12. The composition of claim 10 wherein the polycyclic, heteroatom-containing, hydrocarbon compound comprises phenothiazine, a derivative of phenothiazine, 2-methylthiophenothiazine, 10-methylphenothiazine, monotetradecylphenothiazine, ditetradecylphenothiazine, monodecylphenothiazine, didecylphenothiazine, monononylphenothiazine, dinonylphenothiazine, monoctylphenothiazine, dioctylphenothiazine, monobutylphenothiazine, dibutylphenothiazine, monostyrylphenothiazine, distyrylphenothiazine, butyloctylphenothiazine, styryloctylphenothiazine, and mixtures thereof.

13. The composition of claim 12 which comprises an alkylated phenothiazine, an alkylated derivative of phenothiazine, or mixtures thereof.

14. The composition of claim 9 wherein the one or more alkylated polycyclic, heteroatom-containing, hydrocarbon compounds comprise monoalkylated polycyclic, heteroatom-containing, hydrocarbon compounds and dialkylated polycyclic, heteroatom-containing, hydrocarbon compounds, wherein the ratio of monoalkylated polycyclic, heteroatom-containing, hydrocarbon compounds to dialkylated polycyclic, heteroatom-containing, hydrocarbon compounds is from 5:95 to 95:5.

15. The composition of claim 9 wherein the alkylated polycyclic, heteroatom-containing, hydrocarbon compounds have a quaternary carbon content from 50 percent to 100 percent, based on the total primary, secondary, tertiary and quaternary carbons in the alkylated polycyclic, heteroatom-containing, hydrocarbon compounds.

16. The composition of claim 9 which is selected from a polycyclic, heteroatom-containing, hydrocarbon compound-containing mPAO dimer, trimer, tetramer, pentamer, hexamer and higher oligomer.

17. The composition of claim 9 wherein the process is carried out under reaction conditions sufficient to couple the polyalphaolefin oligomer with at least one polycyclic, heteroatom-containing, hydrocarbon compound, to produce said one or more alkylated polycyclic, heteroatom-containing, hydrocarbon compounds.

18. The composition of claim 9 wherein the catalyst comprises a Bronsted acid, a Lewis acid, an aluminum silicate, an ion exchange resin, a zeolite, a naturally occurring sheet silicate, a modified sheet silicate, and an acidic ionic liquid.

19. A process for producing a composition comprising one or more alkylated polycyclic, heteroatom-containing, hydrocarbon compounds, said process comprising reacting a polyalphaolefin oligomer with at least one polycyclic, heteroatom-containing, hydrocarbon compound, in the presence of a catalyst, under reaction conditions sufficient to produce said composition.

20. The process of claim 19 which is carried out under reaction conditions sufficient to couple the polyalphaolefin oligomer with the at least one polycyclic, heteroatom-containing, hydrocarbon compound, to produce said composition.

21. The process of claim 19 wherein the catalyst comprises a Bronsted acid, a Lewis acid, an aluminum silicate, an ion exchange resin, a zeolite, a naturally occurring sheet silicate, a modified sheet silicate, and an acidic ionic liquid.

22. The process of claim 19 wherein the polyalphaolefin oligomer is selected from a mPAO dimer (C₁₂-C₄₂), trimer (C₁₈-C₄₂), tetramer (C₂₄-C₅₆), pentamer (C₃₀-C₇₀), and hexamer (C₃₆-C₈₄), and the at least one polycyclic, heteroatom-containing, hydrocarbon compound is selected from phenothiazine, phenyl phenothiazine, 2-methylthiophenothiazine, 10-methylphenothiazine, monotetradecylphenothiazine, ditetradecylphenothiazine, monodecylphenothiazine, didecylphenothiazine, monononylphenothiazine, dinonylphenothiazine, monoctylphenothiazine, dioctylphenothiazine, monobutylphenothiazine, dibutylphenothiazine, monostyrylphenothiazine, distyrylphenothiazine, butyloctylphenothiazine, styryloctylphenothiazine, and mixtures thereof.

23. The process of claim 19 wherein the one or more alkylated polycyclic, heteroatom-containing, hydrocarbon compounds are represented by the formula 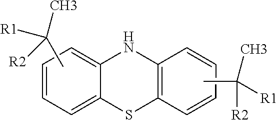
wherein R1 and R2 are same or different and are the residue of an alkyl group having more than 10 carbon atoms wherein the composition has a viscosity (Kv₁₀₀) from 2 to 300 at 100° C. as determined by ASTM D-445, and a viscosity index (VI) from −100 to 300 as determined by ASTM D-2270.

24. The process of claim 23 wherein R1 and R2 are selected from the residue of a mPAO dimer (C₁₂-C₂₈), trimer (C₁₈-C₄₂), tetramer (C₂₄-C₅₆), pentamer (C₃₀-C₇₀), and hexamer (C₃₆-C₈₄).

25. The process of claim 23 wherein the polycyclic, heteroatom-containing, hydrocarbon compound comprises phenothiazine, a derivative of phenothiazine, 2-methylthiophenothiazine, 10-methylphenothiazine, monotetradecylphenothiazine, ditetradecylphenothiazine, monodecylphenothiazine, didecylphenothiazine, monononylphenothiazine, dinonylphenothiazine, monoctylphenothiazine, dioctylphenothiazine, monobutylphenothiazine, dibutylphenothiazine, monostyrylphenothiazine, distyrylphenothiazine, butyloctylphenothiazine, styryloctylphenothiazine, and mixtures thereof.

26. The process of claim 25 wherein the composition comprises an alkylated phenothiazine, an alkylated derivative of phenothiazine, or mixtures thereof.

27. The process of claim 19 wherein the one or more alkylated polycyclic, heteroatom-containing, hydrocarbon compounds comprise monoalkylated polycyclic, heteroatom-containing, hydrocarbon compounds and dialkylated polycyclic, heteroatom-containing, hydrocarbon compounds, wherein the ratio of monoalkylated polycyclic, heteroatom-containing, hydrocarbon compounds to dialkylated polycyclic, heteroatom-containing, hydrocarbon compounds is from 5:95 to 95:5.

28. The process of claim 19 wherein the one or more alkylated polycyclic, heteroatom-containing, hydrocarbon compounds have a quaternary carbon content from 50 percent to 100 percent, based on the total primary, secondary, tertiary and quaternary carbons in the one or more alkylated polycyclic, heteroatom-containing, hydrocarbon compounds.

29. The process of claim 19 wherein the composition is selected from a polycyclic, heteroatom-containing, hydrocarbon compound-containing mPAO dimer, trimer, tetramer, pentamer, hexamer and higher oligomer.

30. A lubricating oil base stock comprising one or more compounds represented by the formula 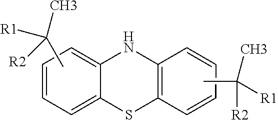
wherein R1 and R2 are same or different and are the residue of an alkyl group having more than 10 carbon atoms wherein the composition has a viscosity (Kv₁₀₀) from 2 to 300 at 100° C. as determined by ASTM D-445, and a viscosity index (VI) from -100 to 300 as determined by ASTM D-2270.

31. The lubricating oil base stock of claim 30 wherein R₁ and R₂ are selected from the residue of a mPAO dimer (C₁₂-C₂₈), trimer (C₁₈-C₄₂), tetramer (C₂₄-C₅₆), pentamer (C₃₀-C₇₀), and hexamer (C₃₆-C₈₄).

32. The lubricating oil base stock of claim 30 wherein the polycyclic, heteroatom-containing, hydrocarbon compound comprises phenothiazine, a derivative of phenothiazine, 2-methylthiophenothiazine, 10-methylphenothiazine, monotetradecylphenothiazine, ditetradecylphenothiazine, monodecylphenothiazine, didecylphenothiazine, monononylphenothiazine, dinonylphenothiazine, monoctylphenothiazine, dioctylphenothiazine, monobutylphenothiazine, dibutylphenothiazine, monostyrylphenothiazine, distyrylphenothiazine, butyloctylphenothiazine, styryloctylphenothiazine, and mixtures thereof.

33. The lubricating oil base stock of claim 30 which comprises one or more alkylated polycyclic, heteroatom-containing, hydrocarbon compounds.

34. The lubricating oil base stock of claim 30 which comprises an alkylated phenothiazine, an alkylated derivative of phenothiazine, or mixtures thereof.

35. The lubricating oil base stock of claim 33 wherein the one or more alkylated polycyclic, heteroatom-containing, hydrocarbon compounds comprise monoalkylated polycyclic, heteroatom-containing, hydrocarbon compounds and dialkylated polycyclic, heteroatom-containing, hydrocarbon compounds, wherein the ratio of monoalkylated polycyclic, heteroatom-containing, hydrocarbon compounds to dialkylated polycyclic, heteroatom-containing, hydrocarbon compounds is from 5:95 to 95:5.

36. The lubricating oil base stock of claim 33 wherein the alkylated polycyclic, heteroatom-containing, hydrocarbon compounds have a quaternary carbon content from 50 percent to 100 percent, based on the total primary, secondary, tertiary and quaternary carbons in the alkylated polycyclic, heteroatom-containing, hydrocarbon compounds.

37. The lubricating oil base stock of claim 30 which is selected from a polycyclic, heteroatom-containing, hydrocarbon compound-containing mPAO dimer, trimer, tetramer, pentamer, hexamer and higher oligomer.

38. A lubricating oil comprising a lubricating oil base stock as a major component, and a polycyclic, heteroatom-containing, hydrocarbon compound cobase stock as a minor component;
wherein said polycyclic, heteroatom-containing, hydrocarbon compound cobase stock comprises one or more compounds represented by the formula 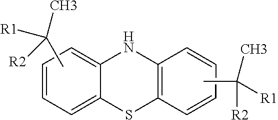
wherein R1 and R2 are same or different and are the residue of an alkyl group having more than 10 carbon atoms wherein the composition has a viscosity (Kv₁₀₀) from 2 to 300 at 100° C. as determined by ASTM D-445, and a viscosity index (VI) from −100 to 300 as determined by ASTM D-2270.

39. The lubricating oil of claim 38 wherein the lubricating oil base stock comprises a Group I, II, III, IV or V base oil stock.

40. The lubricating oil of claim 38 wherein the lubricating oil base stock comprises a polyalphaolefin (PAO) or gas-to-liquid (GTL) oil base stock.

41. The lubricating oil of claim 38 wherein the lubricating oil base stock is present in an amount from 50 weight percent to 99 weight percent, and the polycyclic, heteroatom-containing, hydrocarbon compound cobase stock is present in an amount from 1 weight percent to 50 weight percent, based on the total weight of the lubricating oil.

42. The lubricating oil of claim 38 wherein R₁ and R₂ are selected from the residue of a mPAO dimer (C₁₂-C₂₈), trimer (C₁₈-C₄₂), tetramer (C₂₄-C₅₆), pentamer (C₃₀-C₇₀), and hexamer (C₃₆-C₈₄).

43. The lubricating oil of claim 38 wherein the polycyclic, heteroatom-containing, hydrocarbon compound comprises phenothiazine, a derivative of phenothiazine, 2-methylthiophenothiazine, 10-methylphenothiazine, monotetradecylphenothiazine, ditetradecylphenothiazine, monodecylphenothiazine, didecylphenothiazine, monononylphenothiazine, dinonylphenothiazine, monoctylphenothiazine, dioctylphenothiazine, monobutylphenothiazine, dibutylphenothiazine, monostyrylphenothiazine, distyrylphenothiazine, butyloctylphenothiazine, styryloctylphenothiazine, and mixtures thereof.

44. The lubricating oil of claim 38 wherein said polycyclic, heteroatom-containing, hydrocarbon compound cobase stock comprises one or more alkylated polycyclic, heteroatom-containing, hydrocarbon compounds.

45. The lubricating oil of claim 38 wherein said polycyclic, heteroatom-containing, hydrocarbon compound cobase stock comprises an alkylated phenothiazine, an alkylated derivative of phenothiazine, or mixtures thereof.

46. The lubricating oil of claim 44 wherein the one or more alkylated polycyclic, heteroatom-containing, hydrocarbon compounds comprise monoalkylated polycyclic, heteroatom-containing, hydrocarbon compounds and dialkylated polycyclic, heteroatom-containing, hydrocarbon compounds, wherein the ratio of monoalkylated polycyclic, heteroatom-containing, hydrocarbon compounds to dialkylated polycyclic, heteroatom-containing, hydrocarbon compounds is from 5:95 to 95:5.

47. The lubricating oil of claim 44 wherein the alkylated polycyclic, heteroatom-containing, hydrocarbon compounds have a quaternary carbon content from 50 percent to 100 percent, based on the total primary, secondary, tertiary and quaternary carbons in the alkylated polycyclic, heteroatom-containing, hydrocarbon compounds.

48. The lubricating oil of claim 38 wherein said polycyclic, heteroatom-containing, hydrocarbon compound cobase stock is selected from a polycyclic, heteroatom-containing, hydrocarbon compound-containing mPAO dimer, trimer, tetramer, pentamer, hexamer and higher oligomer.

49. The lubricating oil of claim 38 wherein the polycyclic, heteroatom-containing, hydrocarbon compound cobase stock comprises a polycyclic, heteroatom-containing, hydrocarbon compound-containing polyalphaolefin oligomer.

50. The lubricating oil of claim 38 wherein the polycyclic, heteroatom-containing, hydrocarbon compound cobase stock is formed from the reaction of a polyalphaolefin dimer, trimer or tetramer with phenothiazine or derivative of phenothiazine.

51. The lubricating oil of claim 38 wherein the lubricating oil further comprises one or more of a viscosity improver, antioxidant, detergent, dispersant, pour point depressant, corrosion inhibitor, metal deactivator, seal compatibility additive, anti-foam agent, inhibitor, and anti-rust additive.

52. A method for improving oxidative stability of a lubricating oil by using as the lubricating oil a formulated oil comprising a lubricating oil base stock as a major component, and polycyclic, heteroatom-containing, hydrocarbon compound cobase stock as a minor component; wherein said polycyclic, heteroatom-containing, hydrocarbon compound cobase stock comprises one or more compounds represented by the formula 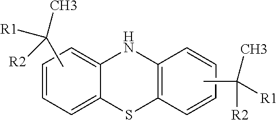
wherein R1 and R2 are same or different and are the residue of an alkyl group having more than 10 carbon atoms wherein the composition has a viscosity (Kv₁₀₀) from 2 to 300 at 100° C. as determined by ASTM D-445, and a viscosity index (VI) from −100 to 300 as determined by ASTM D-2270. and an oxidation onset temperature (OOT)/oxidation peak temperature (OPT) ranging from 175° C./325° C. to 225° C./375° C.; wherein oxidative stability is improved as compared to oxidative stability achieved using a lubricating engine oil containing a minor component other than the polycyclic, heteroatom-containing, hydrocarbon compound cobase stock.

53. A composition comprising one or more compounds represented by the formula 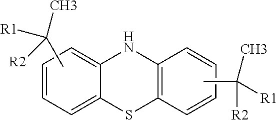
wherein R1 and R2 are same or different and are the residue of an alkyl group having more than 10 carbon atoms wherein the composition has a viscosity (Kv₁₀₀) from 2 to 300 at 100° C. as determined by ASTM D-445, and a viscosity index (VI) from −100 to 300 as determined by ASTM D-2270.

54. A lubricating oil base stock comprising one or more compounds represented by the formula 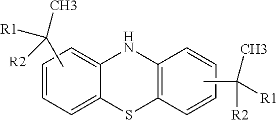
wherein R1 and R2 are same or different and are the residue of an alkyl group having more than 10 carbon atoms wherein the composition has a viscosity (Kv₁₀₀) from 2 to 300 at 100° C. as determined by ASTM D-445, and a viscosity index (VI) from −100 to 300 as determined by ASTM D-2270.

55. The composition of claim 1 wherein the polycyclic, heteroatom-containing, hydrocarbon compound is represented by the formula 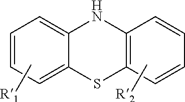
wherein R′₁ and R′₂ are the same or different and are hydrogen or the residue of an alkyl group having from 1 to 40 carbon atoms.

56. The lubricating oil base stock of claim 30 wherein the polycyclic, heteroatom-containing, hydrocarbon compound is represented by the formula 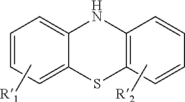
wherein R′₁ and R′₂ are the same or different and are hydrogen or the residue of an alkyl group having from 1 to 40 carbon atoms.

57. A lubricating oil comprising the lubricating oil base stock of claim 30 which is present in an amount from 20 weight percent to 80 weight percent, based on the total weight of the lubricating oil.
